SEQUENCE LISTING (25 sequences)

SEQ ID NO: 1 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 2 - protein, 12 aa, Homo sapiens
EPKSCDKTHTCP

SEQ ID NO: 3 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 330 aa, Homo sapiens [sequence omitted]

SEQ ID NO: 6 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 107 aa, Homo sapiens [sequence omitted]

SEQ ID NO: 8 - protein, 8 aa, Artificial Sequence
  2A peptide consensus sequence
  VARIANT 2: Xaa may be any amino acid
  VARIANT 4: Xaa may be any amino acid
DXEXNPGP

SEQ ID NO: 9 - protein, 13 aa, Artificial Sequence
  2A peptide consensus sequence
  VARIANT 2: Xaa may be any amino acid
  VARIANT 3: Xaa may be any amino acid
  VARIANT 4: Xaa may be any amino acid
  VARIANT 9: Xaa may be any amino acid
LXXXGDVEXNPGP

SEQ ID NO: 10 - protein, 24 aa, Artificial Sequence
  2A peptide of foot-and-mouth disease virus
APVKQTLNFDLLKLAGDVESNPGP

SEQ ID NO: 11 - protein, 33 aa, Artificial Sequence
  2A peptide of foot-and-mouth disease virus
EARHKQKIVAPVKQTLNFDLLKLAGDVESNPGP

SEQ ID NO: 12 - protein, 19 aa, Artificial Sequence
  2A peptide of porcine teschovirus-1
ATNFSLLKQAGDVEENPGP

SEQ ID NO: 13 - protein, 20 aa, Artificial Sequence
  2A peptide of equine rhinitis A virus
QCTNYALLKLAGDVESNPGP

SEQ ID NO: 14 - protein, 18 aa, Artificial Sequence
  2A peptide of Thosea asigna virus
EGRGSLLTCGDVEENPGP

SEQ ID NO: 15 - protein, 20 aa, Artificial Sequence
  2A peptide of Thosea asigna virus
RAEGRGSLLTCGDVEENPGP

SEQ ID NO: 16 - protein, 25 aa, Strongylocentrotus purpuratus
DGFCILYLLLILLMRSGDVETNPGP

SEQ ID NO: 17 - protein, 20 aa, Amphimedon queenslandica
LLCFMLLLLLSGDVELNPGP

SEQ ID NO: 18 - protein, 20 aa, Amphimedon queenslandica
HHFMFLLLLLAGDIELNPGP

SEQ ID NO: 19 - protein, 19 aa, Saccoglossus kowalevskii
WFLVLLSFILSGDIEVNPG

SEQ ID NO: 20 - protein, 20 aa, Branchiostoma floridae
KNCAMYMLLLSGDVETNPGP

SEQ ID NO: 21 - protein, 20 aa, Branchiostoma floridae
MVISQLMLKLAGDVEENPGP

SEQ ID NO: 22 - protein, 4 aa, Artificial Sequence
  furin recognition site
  VARIANT 2: Xaa may be any amino acid
  VARIANT 3: Xaa may be Arg or Lys
RXXR

SEQ ID NO: 23 - protein, 4 aa, Artificial Sequence
  furin recognition site
RKRR

SEQ ID NO: 24 - protein, 4 aa, Artificial Sequence
  furin recognition site
RRKR

SEQ ID NO: 25 - protein, 37 aa, Artificial Sequence
  furin/2A peptide linker
RKRREARHKQKIVAPVKQTLNFDLLKLAGDVESNPGP

(19) United States
(12) Patent Application Publication
(10) Pub. No.: US 2025/0282894 A1
(43) Pub. Date: Sep. 11, 2025

(54) EXPRESSION TECHNOLOGY FOR ANTIBODY CONSTRUCTS

(71) Applicant: Novartis AG, Basel (CH)

(72) Inventors: Dattananda CHELUR, Chesterbrook, PA (US); Martin HESSLING, Biberach (DE); Anett RITTER, Weil am Rhein (DE)

(21) Appl. No.: 18/253,903

(22) PCT Filed: Nov. 22, 2021
(86) PCT No.: PCT/IB2021/060796

§ 371 Date: May 22, 2023

Related U.S. Application Data
(60) Provisional application No. 63/117,379, filed on Nov. 23, 2020.

(51) Int. Cl. C07K 16/46; C12N 15/85

(52) U.S. Cl. CPC C07K 16/468; C12N 15/85; C07K 2317/31; C07K 2317/522; C07K 2317/524; C07K 2317/526; C07K 2317/53; C07K 2317/622

(57) ABSTRACT

The present invention pertains to expression vector designs for antibody constructs. Different polypeptide chains of an antibody construct are encoded within the same open reading frame, connected to each other by a 2A peptide linker. This expression vector design leads to homogeneous expression and correct assembly of the antibody construct.

### FIELD OF THE INVENTION

The present invention pertains to the field of recombinant protein production. New expression strategies for antibody constructs are provided. In particular, nucleic acid products are provided for production of antibody constructs with several different polypeptide chains, wherein the polypeptide chains are encoded within the same open reading frame, separated by 2A peptides which result in the generation of separate polypeptide chains. Thereby, homogeneous expression and correct assembly of the antibody construct is achieved.

### BACKGROUND OF THE INVENTION

Bispecific antibodies are antibodies which bind to two distinct epitopes. Most commonly, they are constructed by pairing a heavy chain-light chain pair directed against a first epitope with another heavy chain-light chain pair directed against a second epitope. Such antibody constructs are well known in the art. One approach for generating bispecific antibodies is the so called knob-into-hole (KiH) technology described e.g. by Ridgway et al. (1996) Protein Engineering 9(7): 617-621. Herein, the first heavy chain is modified to display a hole like structure by substituting larger amino acids with smaller amino acids, and the second heavy chain is modified to display a knob like structure at the corresponding site in the heavy chain:heavy chain interface, using amino acid substitutions where a smaller amino acid is replaced by a larger amino acid. Since pairing of knob and hole heavy chains is favored bispecific antibodies are formed, i.e. hetero-tetrameric proteins consisting of two different light and two different heavy chains.

However, incorrect assembly of the antibody, especially due to mispairing of a light chain with the wrong heavy chain or pairing of two identical heavy chains, or incomplete constructs missing one polypeptide chain still occurs in the host cell. In addition, the polypeptide chains of a bispecific antibody are often expressed in different amounts, for example because the expression cassettes of the polypeptide chains are expressed in differing amounts, leading to imbalanced mRNA levels. This increases the amount of incorrectly assembled antibody constructs.

The incorrectly assembled antibodies reduce the overall yield of the production process and are particularly difficult to remove during the purification process.

Thus, existing methods for generating bispecific antibodies might not provide sufficient overall yield, purity and product quality at a sufficiently cost effective way to enable manufacturing in a scale feasible for clinical development and commercialization. In addition, any modification of protein chains inherently increases the risk to induce anti-drug antibodies. Therefore, approaches which only require minimal protein engineering might be clinically favorable.

In view of the above, there is a need in the art to provide improved strategies to produce bispecific antibodies and other antibody constructs which result in a high yield and ratio of correctly assembled antibody constructs.

### SUMMARY OF THE INVENTION

The present inventors have found that antibody constructs which comprise three or more different polypeptide chains such as bispecific antibodies or antibody fusion proteins can be produced with significantly increased efficacy if at least some of the different polypeptide chains are encoded within the same open reading frame using a self-cleaving peptide linker such as a 2A peptide between said polypeptide chains. This approach was shown by the inventors to provide a balanced expression on mRNA level which thereby leads also to a more balanced protein level. Furthermore, translation of different polypeptide chains from one mRNA occurs in close proximity in the host cells. Thereby, the produced antibody constructs are correctly assembled and unwanted mispairing of the polypeptide chains as well as excess production of some of the polypeptide chains is prevented. The present invention therefore provides for an increased ratio of correctly formed antibody constructs expressed by the host cells and thus, an increased overall yield and purity of the desired product. The inventors were able to demonstrate the advantages of the approach according to the present invention for several different antibody constructs which all showed a much higher rate of correctly assembled proteins compared to the conventional approach.

Therefore, in a first aspect, the present invention is directed to a nucleic acid product consisting of one or more vector nucleic acids encoding an antibody construct comprising at least three different polypeptide chains, wherein the antibody construct comprises an antibody heavy chain, and wherein at least two different polypeptide chains of the antibody construct are encoded within the same open reading frame, wherein consecutive polypeptide chains encoded within said open reading frame are connected by a peptide linker comprising a 2A peptide.

In a second aspect, the present invention provides a host cell comprising the nucleic acid product according to the first aspect.

In a third aspect, the present invention provides a method for producing an antibody construct, comprising the steps of
(a) providing a host cell according to the second aspect,
(b) cultivating the host cell in a cell culture under conditions which allow for production of the antibody construct,
(c) obtaining the antibody construct from the cell culture, and
(d) optionally processing the antibody construct.

In a fourth aspect, the present invention provides the use of the nucleic acid product according the first aspect or the host cell according to the second aspect for the production of an antibody construct.

In a fifth aspect, the present invention provides a method for producing a host cell according to the second aspect, comprising introducing the nucleic acid product according to the first aspect into a host cell.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping. In specific embodiments, a nucleic acid refers to a double-stranded deoxyribonucleic acid.

A “nucleic acid product” according to the present invention is a nucleic acid or a set of two or more nucleic acids which together code for a desired polypeptide or protein. In particular, a nucleic acid product which codes for a protein comprised of two or more different polypeptide chains includes nucleic acid products which consist of one nucleic acid coding for all of the different polypeptide chains, as well as nucleic acid products which consist of two or more nucleic acids, wherein each of these nucleic acids codes for at least one of the different polypeptide chains and all nucleic acids of the nucleic acid product together code for all of the different polypeptide chains of the protein. Different nucleic acids of a nucleic acid product are generally designed to harmonize with each other. For example, the different nucleic acids may have different selection markers so that maintenance of each nucleic acid in a transfected host cell can be controlled.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA. The vector according to the present invention may be present in circular or linearized form. A “vector nucleic acid” as used herein is a nucleic acid which forms a vector or is the nucleic acid part of a vector.

The terms “5′” and “3′” is a convention used to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

A “polypeptide” or “polypeptide chain” refers to a molecule comprising a polymer of amino acids linked together by peptide bonds. Polypeptides include polypeptides of any length, including proteins (for example, having more than 50 amino acids) and peptides (for example, having 2-49 amino acids). Especially, a polypeptide or polypeptide chain can be a part of a protein which consists of two or more polypeptide chains. Polypeptides include proteins and/or peptides of any activity or bioactivity. The polypeptide can be a pharmaceutically or therapeutically active compound, or a research tool to be utilized in assays and the like. Suitable examples are outlined below.

A target amino acid sequence is “derived” from or “corresponds” to a reference amino acid sequence if the target amino acid sequence shares an identity over its entire length with the reference amino acid sequence of at least 75%, more preferably at least 80%, at least 85%, at least 90%, at least 93%, at least 95%, at least 97%, at least 98% or at least 99%. In particular embodiments, a target amino acid sequence which is “derived” from or “corresponds” to a reference amino acid sequence is 100% homologous, or in particular 100% identical, over its entire length with the reference amino acid sequence. Similarly, a target nucleotide sequence is “derived” from or “corresponds” to a reference nucleotide sequence if the target nucleotide sequence shares an identity over its entire length with the reference nucleotide sequence of at least 75%, more preferably at least 80%, at least 85%, at least 90%, at least 93%, at least 95%, at least 97%, at least 98% or at least 99%. In particular embodiments, a target nucleotide sequence which is “derived” from or “corresponds” to a reference nucleotide sequence is 100% identical over its entire length with the reference nucleotide sequence. An “identity” of an amino acid sequence or nucleotide sequence is preferably determined according to the invention over the entire length of the reference sequence.

The term “antibody” in particular refers to an antibody protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The amino acid sequences of the human CH1, hinge region, CH2 and CH3 of the γ1-type heavy chain are shown in SEQ ID NOs: 1 to 4, respectively, and the entire constant region of the human γ1-type heavy chain is shown in SEQ ID NO: 5. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. The amino acid sequences of the constant domain CL of the human λ-type and κ-type light chain are shown in SEQ ID NOs: 6 and 7, respectively. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. The antibody can be e.g. a humanized, human or chimeric antibody.

The term “antibody” as used herein also includes fragments, derivatives and engrafts of said antibody. A “fragment or derivative” of an antibody in particular is a protein or glycoprotein which is derived from said antibody and is capable of binding to the same antigen, in particular to the same epitope as the antibody. In further embodiments, a “fragment or derivative” of an antibody especially refers to polypeptides or proteins which comprise one or more Fc regions of an antibody, and may or may not comprise an antigen binding region. Thus, a fragment or derivative of an antibody herein generally refers to a functional fragment or derivative, where the function of the antibody is binding of an antigen and/or interaction with Fc receptors. An “engraft” of an antibody especially refers to said antibody wherein a heterologous polypeptide is introduced into or (partially) replaces a CDR sequence of the antibody. An exemplary antibody engraft is described in US 2017/0158747 A1.

The term “antibody construct” as used herein refers to any protein which comprises at least one protein domain derived from an antibody. In particular, an antibody construct is an artificial protein and may comprise parts of different natural or genetically engineered proteins, including at least one antibody. Especially, an antibody construct comprises at least one immunoglobulin domain derived from an antibody, in particular from a human IgG antibody. In certain embodiments, the antibody construct comprises at least a constant immunoglobulin domain derived from an antibody, such as a CH2 domain or a CH3 domain, especially a CH2 domain and a CH3 domain.

The cells referred to herein in particular are host cells. According to the invention, the term “host cell” relates to any cell which can be transformed or transfected with an exogenous nucleic acid. Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

A “homogeneous production” of the polypeptide chains refers to a balanced level of the polypeptide chains encoded within the same open reading frame obtained after translation due to a balanced level of the encoding mRNA obtained after transcription.

The term “pharmaceutical composition” or “pharmaceutical formulation” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The numbers given herein are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of new expression vectors for antibody constructs. Different polypeptide chains of the antibody constructs are encoded on these vectors in one open reading frame. The polypeptide chains are each connected in this open reading frame via a peptide linker comprising a 2A peptide. 2A peptides are “self-cleaving” peptides which automatically result in separate polypeptide chains after translation. The open reading frame encoding the different polypeptide chains is transcribed into one mRNA, which then is translated and automatically cleaved into the different polypeptide chains.

Amino acids of the 2A peptide linker which remain on the C terminus of the N terminal polypeptide chain can optionally be removed by incorporating a specific protease cleavage site between the N terminal polypeptide chain and the 2A peptide and using a respective protease, for example furin. Advantageously, the N terminal polypeptide chain is an antibody heavy chain. In this case, the residual, C terminal amino acids of the linker peptide are removed when the natural C terminal lysine residue of the heavy chain is cleaved of by cellular carboxypeptidases.

In order to get rid of the residual amino acids of the 2A peptide which remain on the N terminus of the C terminal polypeptide chain, said polypeptide chain may be encoded including a signal peptide. The signal peptide is cleaved off by the cellular processes of protein maturation and thereby also removes the residual amino acids of the 2A peptide.

The above approach is highly advantageous for producing antibody constructs, especially constructs which comprise three or more different polypeptide chains. The present inventors could demonstrate that balanced expression is greatly increased by using respective expression constructs. The different polypeptide chains are translated from the same mRNA and therefore are generally produced in equimolar amounts. Thereby, unwanted surplus production of only some of the polypeptide chains due to imbalanced expression can be avoided. In addition, translation of an mRNA coding for several different polypeptide chains using the 2A peptide technology results in the production of these polypeptide chains in close proximity in the cell since it is believed that they are produced by the same ribosome. This markedly enhances correct chain pairing and protein assembly, leading to a higher relative amount of correctly formed antibody constructs and less unwanted side products such as complexes with the wrong or missing polypeptide chains. Thereby, purification of the desired antibody construct is eased and ultimately the overall yield is improved.

### 1. NUCLEIC ACID PRODUCTS ENCODING AN ANTIBODY CONSTRUCT

In view of these findings, the present invention provides in a first aspect a nucleic acid product consisting of one or more vector nucleic acids encoding an antibody construct comprising at least three different polypeptide chains, wherein the antibody construct comprises an antibody heavy chain, and wherein at least two different polypeptide chains of the antibody construct are encoded within the same open reading frame, wherein consecutive polypeptide chains encoded within said open reading frame are connected by a peptide linker comprising a 2A peptide.

In particular, the nucleic acid product provides for a more homogeneous cellular production of the polypeptide chains of the antibody construct compared to production of the polypeptide chains of the antibody construct using a nucleic acid product wherein each polypeptide chain of the antibody construct is encoded within a separate open reading frame.

The cellular product of the polypeptide chains can be determined on the protein level or on the mRNA level. A more homogeneous cellular production of the polypeptide chains in particular refers to a lower difference between the amounts of each polypeptide chain produced by the cell. Especially, the quotient of the highest amount of a polypeptide chain divided by the lowest amount of a polypeptide chain of the antibody construct as produced by the host cell is lower when using the nucleic acid product according to the present invention compared to a nucleic acid product wherein each polypeptide chain of the antibody construct is encoded within a separate open reading frame. Furthermore, a more homogeneous cellular production of the polypeptide chains for example refers to a lower difference between the amounts of the mRNAs coding for each polypeptide chain produced by the cell. Especially, the quotient of the highest amount of an mRNA coding for a polypeptide chain divided by the lowest amount of an mRNA coding for a polypeptide chain of the antibody construct as produced by the host cell is lower when using the nucleic acid product according to the present invention compared to a nucleic acid product wherein each polypeptide chain of the antibody construct is encoded within a separate open reading frame.

In certain embodiments, the amounts of the different polypeptide chains of said antibody construct in the cell do not differ by more than factor 20, especially not more than factor 10, not more than factor 8 or not more than factor 5. In further embodiments, the amounts of the mRNAs coding for the different polypeptide chains of said antibody in the cell do not differ by more than factor 20, especially not more than factor 10, not more than factor 8 or not more than factor 5.

Furthermore, the nucleic acid product especially provides for a higher relative amount of correctly assembled antibody constructs after expression of the polypeptide chains of the antibody construct compared to the relative amount of correctly assembled antibody constructs after expression of the polypeptide chains of the antibody construct using a nucleic acid product wherein each polypeptide chain of the antibody construct is encoded within a separate open reading frame. In certain embodiments, the relative amount of correctly assembled antibody constructs after expression of the polypeptide chains of the antibody construct is at least 5 percentage points, especially at least 10 percentage points, at least 15 percentage points or at least 20 percentage points, higher than the relative amount of correctly assembled antibody constructs using a nucleic acid product wherein each polypeptide chain is encoded within a separate open reading frame.

The comparison with a nucleic acid product wherein each polypeptide chain is encoded within a separate open reading frame is in particular done with the same or highly similar conditions and production means, especially with the same coding sequences, the same promoters, the same host cell line, and at the same culture conditions.

### 1.1 the Nucleic Acid Product

The nucleic acid product encompasses one or more open reading frames which together code for all polypeptide chains of the antibody construct. In case the polypeptide chains of the antibody construct are encoded by more than one open reading frame, these open reading frames may be present on the same or on different vector nucleic acids. In certain embodiments, the nucleic acid product consists of one vector nucleic acid encoding the antibody construct. In these embodiments, the one or more open reading frames coding for all polypeptide chains of the antibody construct are present on this vector nucleic acid. In other embodiments, the nucleic acid product consists of two or more, in particular two, vector nucleic acids encoding the antibody construct. In these embodiments, polypeptide chains of the antibody construct are generally encoded by two or more open reading frames, which are present on the two or more vector nucleic acids. One vector nucleic acid may comprise one or more open reading frames coding for polypeptide chains of the antibody construct. The use of only one vector nucleic acid is preferred.

Different polypeptide chains of the antibody construct which are encoded in the same open reading frame are connected by a peptide linker comprising a 2A peptide. In embodiments wherein more than two polypeptide chains of the antibody construct are encoded in the same open reading frame, a peptide linker comprising a 2A peptide connects consecutive polypeptide chains.

In certain embodiments, the open reading frame coding for two or more polypeptide chains of the antibody construct is part of an expression cassette which enables expression of the open reading frame. In case the polypeptide chains of the antibody construct are encoded by more than one open reading frame, the different open reading frames may be part of the same expression cassette or of separate expression cassettes. In embodiments wherein two or more open reading frames are part of the same expression cassette, consecutive open reading frames are in particular connected by an internal ribosome entry site (IRES).

An expression cassette in particular comprises, in addition to the open reading frame(s), a promoter operatively linked to the open reading frame(s). The promoter used in the expression cassettes may be any promoter suitable for driving expression in a mammalian host cell. The promoter may for example be selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter and β-actin promoter optionally coupled with CMV early enhancer (CAGG). Specific examples of promoters include cytomegalovirus immediate-early promoter, simian virus 40 early promoter, human Ubiquitin C promoter, human elongation factor 1α promoter, mouse phosphoglycerate kinase 1 promoter, Rous sarcoma virus long terminal repeat promoter and chicken β-Actin promoter coupled with CMV early enhancer. In specific embodiments, the promoter is a CMV promoter.

In embodiments wherein the open reading frames coding for the polypeptide chains of the antibody construct are expressed by more than one expression cassette, the expression cassettes preferable comprise similar expression regulation elements, especially the promoters with similar strength. Different promoters or identical promoters may be used, and in particular different promoters with similar strength are used.

The vector nucleic acid may comprise further elements such as a marker gene and an origin or replication. In some embodiments, the vector nucleic acid is suitable for stable transfection of a host cell, especially a mammalian host cell such as a rodent or human cell, especially a CHO cell. In particular, the vector nucleic acid is a plasmid.

In some embodiments, each vector nucleic acid of the nucleic acid product comprises at least one selectable marker gene. The selectable marker gene in particular is a mammalian selectable marker gene which allows the selection of mammalian host cells comprising said gene and thus of mammalian host cells comprising the vector nucleic acid.

Non-limiting examples of mammalian selectable marker genes include antibiotic resistance genes e.g. conferring resistance to G418; hygromycin (hyg or hph, commercially available from Life Technologies, Inc. Gaithesboro, Md.); neomycin (neo, commercially available from Life Technologies, Inc. Gaithesboro, Md.); zeocin (Sh Ble, commercially available from Pharmingen, San Diego Calif.); puromycin (pac, puromycin-N-acetyl-transferase, available from Clontech, Palo Alto Calif.), ouabain (oua, available from Pharmingen) and blasticidin (available from Invitrogen). Further suitable selectable marker genes include folate receptor genes such as the folate receptor alpha gene, or genes encoding fluorescent proteins such as GFP and RFP. Respective mammalian selectable marker genes are well known and allow the selection of mammalian cells comprising said genes and thus of cells comprising the vector. Systems using a folate receptor gene are described in WO 2009/080759 and WO 2015/015419. The term “gene” as used herein also refers to a natural or synthetic polynucleotide encoding a functional variant of the selectable marker providing the intended resistance. Hence, also truncated or mutated versions of a wild type gene or synthetic polynucleotides are encompassed as long as they provide the intended resistance.

In some embodiments, the mammalian selectable marker genes may be amplifiable and allow selection of vector-containing mammalian host cells as well as gene amplification. A non-limiting example for an amplifiable, selectable mammalian marker gene is the dihydrofolate reductase (DHFR) gene. Other systems currently in use are among others the glutamine synthetase (gs) system and the histidinol driven selection system. These amplifiable markers are also selectable markers and can thus be used to select those cells that obtained the vector nucleic acid. With amplifiable systems such as the DHFR system, expression of a recombinant protein can be increased by exposing the cells to certain agents promoting gene amplification such as antifolates (e.g. methotrexate (MTX)) in case of the DHFR system. A suitable inhibitor for GS promoting gene amplification is methionine sulphoximine (MSX). Exposure to MSX also results in gene amplification.

In embodiments wherein the nucleic acid product comprises two or more vector nucleic acids, the different vector nucleic acids in particular comprise different selectable marker genes.

The vector nucleic acid in particular is suitable for integration into the genome of a host cell. In some embodiments, the host cell is stably transfected with the vector nucleic acid.

In certain embodiments, the vector nucleic acid further comprises a prokaryotic selectable marker gene. Said prokaryotic selectable marker may provide a resistance to antibiotics such as e.g. ampicillin, kanamycin, tetracycline and/or chloramphenicol.

### 1.2 the Antibody Construct

The antibody construct may be any protein which comprises three or more different polypeptide chains wherein at least one of these polypeptide chains is a heavy chain of an antibody. Hence, the different polypeptide chains of the antibody construct encompass at least one antibody heavy chain, also called first heavy chain in the following.

In specific embodiments, the antibody heavy chain is one of the at least two different polypeptide chains of the antibody construct which are encoded within the same open reading frame. In particular, the antibody heavy chain is the N-terminal polypeptide chain encoded within the open reading frame of the nucleic acid product.

The remaining polypeptide chains may be any polypeptide chains as long as they are capable of assembling together to form the antibody construct. In certain embodiments, the further polypeptide chains include at least one antibody light chain and/or at least one further antibody heavy chain which is different from the first heavy chain. In specific embodiments, the polypeptide chains of the antibody construct are all antibody heavy chains and optionally antibody light chains. Exemplary sets of different polypeptide chains of antibody constructs include: two different antibody heavy chains and two different antibody light chains, two different antibody heavy chains and one antibody light chain, one antibody heavy chain and two different antibody light chains, and two different antibody heavy chains and three different antibody light chains.

In certain embodiments, the antibody construct comprises an antibody light chain binding to the first heavy chain. This antibody light chain is also called first light chain in the following. In specific embodiments, the first heavy chain and the first light chain are encoded within the same open reading frame. In these embodiments, the first antibody heavy chain is preferably the N-terminal polypeptide chain encoded within said open reading frame. In particular, the first light chain is the second polypeptide chain encoded within said open reading frame to which the first heavy chain is connected to by a peptide linker comprising a 2A peptide. In these embodiments, the open reading frame preferably codes first for the first heavy chain, then for the peptide linker and then for the first light chain, in the direction of translation, so that the peptide linker connects the C terminus of the heavy chain with the N terminus of the light chain.

In specific embodiments, the first heavy chain comprises a heavy chain variable region, and the first light chain comprises a light chain variable region. In these embodiments, the heavy chain variable region and the light chain variable region may in particular form an antigen binding region. The antigen binding region especially is capable of specifically binding an antigen.

In certain embodiments, the antibody construct comprises a second antibody heavy chain. The second heavy chain in particular is different from the first heavy chain. Preferably, the first heavy chain and the second heavy chain bind to each other in the antibody construct. In specific embodiments, the first heavy chain and the second heavy chain bind to each other using a knob-into-hole technology.

In embodiments wherein the antibody construct comprises a second heavy chain, the second heavy chain may be encoded within the same open reading frame as the first heavy chain. The first heavy chain, the linker peptide and the second heavy chain may be encoded consecutively in this order from 5′ to 3′. In case this open reading frame codes for a further polypeptide chain, such as the first light chain, then said further polypeptide chain may be encoded, for example, between the first and second heavy chains, or after the second heavy chain.

In alternative embodiments wherein the antibody construct comprises a second heavy chain, the second heavy chain is encoded within a second open reading frame different from the open reading frame encoding the first heavy chain. In these embodiments, one or more further polypeptide chains of the antibody construct may be encoded within the second open reading frame. In this case, the N-terminal polypeptide chain encoded within said second open reading frame preferably is the second heavy chain which is connected to a further polypeptide chain encoded within said second open reading frame by a peptide linker comprising a 2A peptide.

In embodiments wherein the antibody construct comprises a second heavy chain, the first heavy chain and the second heavy chain in particular bind to each other using a knob-into-hole technology.

In certain embodiments, the antibody construct comprises a second antibody light chain. In embodiments wherein the antibody construct comprises a second antibody heavy chain and a second antibody light chain, the second light chain in particular is bound to the second heavy chain. In these embodiments, the second heavy chain and the second light chain are preferably encoded within the same open reading frame comprising a peptide linker connecting these two polypeptide chains, wherein the peptide linker comprises a 2A peptide. In particular, said open reading frame codes first for the second heavy chain, then for the peptide linker and then for the second light chain, in the direction of translation, so that the peptide linker connects the C terminus of the heavy chain with the N terminus of the light chain.

In specific embodiments, the second heavy chain comprises a heavy chain variable region, and the second light chain comprises a light chain variable region. In these embodiments, the heavy chain variable region and the light chain variable region may in particular form an antigen binding region. The antigen binding region especially is capable of specifically binding an antigen.

In embodiments wherein the heavy and light chain variable regions of the first heavy and light chain form a first antigen binding region and the heavy and light chain variable regions of the second heavy and light chain form a second antigen binding region, the first antigen binding region in particular is capable of specifically binding a first antigen and the second antigen binding region in particular is capable of specifically binding a second antigen.

### 1.3 Antibody Heavy Chains

A heavy chain of an antibody, also referred to as “heavy chain” or “antibody heavy chain” herein, according to the present invention is a polypeptide chain which comprises at least a part of the heavy chain constant region of an antibody, in particular at least one immunoglobulin domain of the heavy chain constant region (also called heavy chain constant domain (CH)) such as a CH1 domain, a CH2 domain and a CH3 domain.

In preferred embodiments, an antibody heavy chain comprises a CH3 domain. In certain embodiments, the CH3 domain comprises a lysine residue at its C terminus.

A CH3 domain as used herein refers to an antibody heavy chain constant domain which is derived from the CH3 domain of a native human antibody, especially of a native human IgG antibody having a γ-type heavy chain. Similarly, the further immunoglobulin domains (VH, CH1, CH2, VL, CL) as used herein refer to antibody heavy chain or light chain domains which are derived from the respective immunoglobulin domains of a native human antibody, especially of a native human IgG antibody having a γ-type heavy chain and a κ- or λ-type light chain. A hinge region as used herein refers to an antibody heavy chain hinge region which is derived from the hinge region of a native human antibody, especially of a native human IgG antibody having a γ-type heavy chain. The term “derived from” in this respect in particular means that the amino acid sequence of the immunoglobulin domain is at least 90% identical, in particular at least 95% identical to the respective immunoglobulin domain of the native human antibody. The immunoglobulin domains and the hinge region of an antibody such as VH, CH1, CH2, CH3, VL, CL and hinge region are collectively referred to herein as antibody domains.

In certain embodiments, an antibody heavy chain comprises a CH2 domain, especially a CH2 domain and a CH3 domain. In specific embodiments, an antibody heavy chain comprises a hinge region, a CH2 domain and a CH3 domain. The antibody heavy chain in particular is capable of binding to another antibody heavy chain and especially of forming a homodimer with an identical antibody heavy chain or a heterodimer with another antibody heavy chain.

In specific embodiments, an antibody heavy chain comprises one or more amino acid substitutions which render it suitable for the knob-into-hole technology. According to this well-known technology two different heavy chains are mutated to form a “knob” in one heavy chain and a corresponding “hole” in the other heavy chain. These knobs and holes are formed by introducing large, bulky amino acids as knobs and small amino acids as holes at positions which are in contact with each other in the antibody. Suitable “knob” amino acids are for example tyrosine and tryptophan and suitable “hole” amino acids are for example alanine, serine, threonine and valine. The amino acid substitutions are generally present in the CH3 domain as this domain provides the main contact site between two the two heavy chains of an antibody. It is also possible to use two or three “knobs” and the corresponding number of “holes”, wherein one heavy chain may comprise both, at least one knob and at least one hole. The knob-into-hole technology is well known and established in the art (see, e.g., Ridgway et al. (1996) Protein Engineering 9(7): 617-621). Suitable mutations include T366W, T366Y and T394W as knobs and T366S, L368A, F405A, Y407V and Y407T as holes, with exemplary knob/hole pairs being T366Y/Y407T and F405A/T394W.

In certain embodiments, an antibody heavy chain comprises a CH1 domain and/or a heavy chain variable domain (VH). In particular, the antibody heavy chain comprises the CH1 domain and/or the VH domain in addition to the CH2 domain, the CH3 domain and optionally the hinge region.

The antibody domains of the heavy chain—so far as present—in particular are arranged in their natural order as found in native antibodies, namely in the order from N terminal to C terminal of VH domain, CH1 domain, hinge region, CH2 domain and CH3 domain.

In certain embodiments, an antibody heavy chain comprises one or more further polypeptide moieties in addition to the antibody domains. These further polypeptide moieties may be positioned anywhere in the heavy chain, for example at the N terminus, at the C terminus or in between two of the antibody domains. Specifically, the further polypeptide moieties are positioned so that they do not interfere with binding of the heavy chain to another heavy chain or to a light chain.

In certain embodiments, the antibody heavy chain is encoded with a signal peptide at the N terminus. The signal peptide is a signal for the host cell to secrete the produced polypeptide chain and is cleaved off after translation. In particular, the antibody heavy chain is encoded with a signal peptide if it is the first polypeptide chain encoded in the open reading frame.

In embodiments wherein the antibody construct comprises at least two different antibody heavy chains, these heavy chains may each independently have one or more of the features described above. Different antibody heavy chains differ from each other in at least one amino acid.

### 1.4 Antibody Light Chains

A light chain of an antibody, also referred to as “light chain” or “antibody light chain” herein, according to the present invention is a polypeptide chain which comprises at least a part of the light chain constant region of an antibody, in particular at least an immunoglobulin domain of the light chain constant region, also called light chain constant domain (CL).

In preferred embodiments, an antibody light chain comprises a CL domain, especially a CL domain derived from the CL domain of a native human antibody, especially of a native human antibody having a κ- or λ-type light chain.

In certain embodiments, an antibody light chain comprises a light chain variable domain (VL). In particular, the antibody light chain comprises the VL domain in addition to the CL domain. The antibody light chain in particular is capable of binding to an antibody heavy chain and especially of forming a heterodimer with an antibody heavy chain.

The antibody domains of the light chain—so far as present—in particular are arranged in their natural order as found in native antibodies, namely in the order from N terminal to C terminal of VL domain and CL domain.

In embodiments wherein the antibody construct comprises at least two different antibody light chains and at least two different antibody heavy chains, one or more amino acid residues at the interface between heavy and light chain may be mutated in the antibody heavy and/or light chains so as to increase correct pairing of heavy and light chains. In particular, one or both of the light chains and/or one or both of the heavy chains are engineered so that each light chain strongly favors its cognate heavy chain. Respective technologies are known in the art. For example, a knob-into-hole technology as described herein for the pairing of two heavy chains can be employed. Alternatively or additionally, an electrostatic steering mechanism can be used, wherein the charge distribution on the interaction surface between light chain and heavy chain are engineered by amino acid substitution so that repelling electrostatic forces reduce or prevent binding of a light chain to the wrong heavy chain. In specific embodiments, the VH and CH1 domains of the heavy chains and the VL and CL domains of the light chains are engineered so that correct chain paring is improved. In alternative embodiments the VH and CH1 domains of the heavy chains and the VL and CL domains of the light chains are not engineered for improving correct chain paring.

In certain embodiments, an antibody light chain comprises one or more further polypeptide moieties in addition to the antibody domains. These further polypeptide moieties may be positioned anywhere in the light chain, for example at the N terminus, at the C terminus or in between two of the antibody domains. Specifically, the further polypeptide moieties are positioned so that they do not interfere with binding of the light chain to a heavy chain.

In certain embodiments, the antibody light chain is encoded with a signal peptide at the N terminus.

In embodiments wherein the antibody construct comprises at least two different antibody light chains, these light chains may each independently have one or more of the features described above. Different antibody light chains differ from each other in at least one amino acid.

### 1.5 Further Polypeptide Moieties

The antibody heavy chains and antibody light chains of the antibody construct may comprise further polypeptide moieties in addition to any antibody domains. These further polypeptide moieties may be any polypeptides fused to the antibody domains.

The further polypeptide moieties in particular have specific functions such as binding to a target molecule. Examples of further polypeptide moieties include single chain antibody fragments such as single chain Fv fragments (scFv) which consists of a heavy chain variable domain and a light chain variable domain linked together by a peptide linker and forming an antigen binding site, and single domain antibody fragments (sdAb) which consist of a heavy chain variable domain especially derived from heavy-chain antibodies found in camelids. In particular, the further polypeptide moieties may provide for an antigen binding region. Further examples of the polypeptide moieties include ligands, cytokines such as interleukins, cell adhesion molecules, growth factors, and functional fragments and constitutively active or dominant negative mutants of these moieties. In certain embodiments, the further polypeptide moieties are capable of binding to and/or activating or inhibiting immune cells such as T cells. The polypeptide moiety may also be a small peptide, for example a peptide toxin.

### 1.6 Peptide Linker

The polypeptide chains encoded in the same open reading frame are connected by a peptide linker comprising a 2A peptide. The peptide linker connects the C terminus of the preceding polypeptide chain with the N terminus of the next polypeptide chain.

The 2A peptide in the peptide linker is a “self-cleaving” peptide. “Self-cleavage” occurs co-translationally so that after translation an N terminal polypeptide chain and a C terminal polypeptide chain are obtained. The underlying self-cleaving mechanism is not yet fully understood and may for example be based on cleavage of the peptide bond directly after translation or skipping of the ribosome so that the peptide bond is not formed in the first place. In certain embodiments, the 2A peptide is derived from a virus selected from the group consisting of foot-and-mouth disease virus, equine rhinitis A virus, porcine teschovirus-1, and Thosea asigna virus. In further embodiments, the 2A peptide is derived from a non-viral organism such as a sea urchin (e.g. Strongylocentrotus purpuratus), a sponge (e.g. Amphimedon queenslandica), an acorn worm (e.g. Saccoglossus kowalevskii) and an amphioxus (e.g. Branchiostoma floridae). In particular, the 2A peptide is derived from foot-and-mouth disease virus.

In specific embodiments, the 2A peptide has an amino acid sequence which comprises the consensus sequence DXEXNPGP (SEQ ID NO: 8), in particular LXXXGDVEXNPGP (SEQ ID NO: 9). The 2A cleavage site is located between the C terminal glycine and proline residues. The 2A peptide in particular comprises an amino acid sequence which is at least 80% identical to one of the amino acid sequences according to SEQ ID NOs: 10 to 21 over their entire length, wherein especially the consensus sequence of SEQ ID NO: 8, in particular the consensus sequence of SEQ ID NO: 9, is conserved. In certain embodiments, the 2A peptide comprises or consists of an amino acid sequence selected from SEQ ID NOs: 10 to 21, in particular the amino acid sequence of SEQ ID NO: 10, especially the amino acid sequence of SEQ ID NO: 11.

In preferred embodiments, the 2A peptide is located at the C terminus of the linker peptide. Especially, the C terminal polypeptide chain is directly fused to the C terminal proline residue of the cleavage site of the 2A peptide. Thereby, the C terminal polypeptide chain is formed after self-cleavage of the 2A peptide with only an additional proline residue at its N terminus.

In certain embodiments, the peptide linker further comprises a protease recognition site N terminal of the 2A peptide. In particular, the protease recognition site is a furin recognition site. The furin recognition site especially has the amino acid sequence of RX(R/K)R (SEQ ID NO: 22), in particular RKRR or RRKR (SEQ ID Nos: 23 and 24). In specific embodiments, the protease recognition site is located at the N terminus of the linker peptide.

The protease recognition site may be directly fused to the 2A peptide or a further linker sequence may be present between protease recognition site and 2A peptide. The further linker sequence may be any sequence, including common linker sequences such as a GS linker, e.g. the amino acid sequence GSG, and sequences derived from the source of the 2A peptide. In certain embodiments, the protease recognition site is directly fused to the 2A peptide and the peptide linker consists of the protease recognition site and the 2A peptide.

The protease recognition site in particular is a recognition site of a protease which is intrinsically expressed in the host cell used for production of the antibody construct. Especially, the protease is ubiquitous expressed in mammalian cells or even in eukaryotic cells. Preferably, the protease is furin which is expressed in almost every eukaryotic cell and especially in any mammalian cell which is used as host cell in protein production. In other embodiments, a certain protease recognition site is used and the host cell is engineered to express the protease recognizing said protease recognition site or the protease recognizing said protease recognition site is added to the antibody construct during or after its production.

For example, the peptide linker may comprise or consist of an amino acid sequence according to SEQ ID NO: 25.

The peptide linker may have any length suitable for linking the different polypeptide chains of the antibody construct and including a self-cleaving 2A peptide and optionally a protease recognition site. In specific embodiments, the peptide linker has a length of 60 amino acids or less, especially 50 amino acids or less, in particular 40 amino acids or less.

In embodiments wherein the open reading frame(s) of the nucleic acid product code for more than one peptide linker, these peptide linkers may each independently have one or more of the features described above and may be different from or identical to each other.

### 1.7 Exemplary Nucleic Acid Products

In one embodiment, the nucleic acid product comprises one or more vector nucleic acids which encode within a first open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide; and
(v) a first light chain comprising a first light chain variable region; and
   which encode within a second open reading frame, in the direction from N terminus to C terminus,
(i) a signal peptide;
(ii) a second heavy chain comprising a second heavy chain variable region;
(iii) a linker peptide comprising a 2A peptide;
(iv) optionally a signal peptide; and
(v) a second light chain comprising a second light chain variable region;
   wherein the first heavy chain variable region and the first light chain variable region form an antigen binding region capable of binding a first antigen; and wherein the second heavy chain variable region and the second light chain variable region form an antigen binding region capable of binding a second antigen; and wherein the first heavy chain and the second heavy chain bind to each other using a knob-into-hole technology.

In one embodiment, the nucleic acid product comprises one vector nucleic acid which encodes within the same open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide;
(v) a first light chain comprising a first light chain variable region;
(vi) a linker peptide comprising a 2A peptide;
(vii) optionally a signal peptide;
(viii) a second heavy chain comprising a second heavy chain variable region;
(ix) a linker peptide comprising a 2A peptide;
(x) optionally a signal peptide; and
(xi) a second light chain comprising a second light chain variable region;
   wherein the first heavy chain variable region and the first light chain variable region form an antigen binding region capable of binding a first antigen; and wherein the second heavy chain variable region and the second light chain variable region form an antigen binding region capable of binding a second antigen; and wherein the first heavy chain and the second heavy chain bind to each other using a knob-into-hole technology.

In one embodiment, the nucleic acid product comprises one or more vector nucleic acids which encode within a first open reading frame, in the direction from N terminus to C terminus,
(i) a signal peptide;
(ii) a first heavy chain comprising a heavy chain variable region;
(iii) a linker peptide comprising a 2A peptide;
(iv) optionally a signal peptide; and
(v) a light chain comprising a light chain variable region; and
   which encode within a second open reading frame, in the direction from N terminus to C terminus,
(i) a signal peptide; and
(ii) a second heavy chain; and
   wherein the heavy chain variable region of the first heavy chain and the light chain variable region form an antigen binding region capable of binding a first antigen; and wherein the second heavy chain comprises an antigen binding region capable of binding a second antigen; and wherein the first heavy chain and the second heavy chain bind to each other using a knob-into-hole technology.

In one embodiment, the nucleic acid product comprises one or more vector nucleic acids which encode within a first open reading frame, in the direction from N terminus to C terminus,
(i) a signal peptide;
(ii) a first heavy chain;
(iii) a linker peptide comprising a 2A peptide;
(iv) optionally a signal peptide; and
(v) a second heavy chain; and
   which encode within a second open reading frame, in the direction from N terminus to C terminus,
(i) a signal peptide; and
(v) a light chain comprising a light chain variable region; and
   wherein
(a) the first heavy chain comprises a heavy chain variable region which forms an antigen binding region with the light chain variable region, capable of binding a first antigen; and the second heavy chain comprises an antigen binding region capable of binding a second antigen; or
(b) the first heavy chain comprises an antigen binding region capable of binding a first antigen; and the second heavy chain comprises a heavy chain variable region which forms an antigen binding region with the light chain variable region, capable of binding a second antigen; and
   wherein the first heavy chain and the second heavy chain bind to each other using a knob-into-hole technology.

In one embodiment, the nucleic acid product comprises one vector nucleic acid which encodes within the open reading frame, in the direction from N terminus to C terminus,
(i) a signal peptide;
(ii) a first heavy chain;
(iii) a linker peptide comprising a 2A peptide;
(iv) optionally a signal peptide;
(v) a second heavy chain comprising a heavy chain variable region;
(vi) a linker peptide comprising a 2A peptide;
(vii) optionally a signal peptide; and
(viii) a light chain comprising a light chain variable region; and
   wherein the first heavy chain comprises an antigen binding region capable of binding a first antigen; and wherein the heavy chain variable region of the second heavy chain and the light chain variable region form an antigen binding region capable of binding a second antigen; and wherein the first heavy chain and the second heavy chain bind to each other using a knob-into-hole technology.

### 1.8 Further Nucleic Acid Products

In a further aspect, the present invention provides a nucleic acid product consisting of one or more vector nucleic acids encoding an antibody construct comprising a first antibody heavy chain and a second antibody heavy chain which is different from the first heavy chain, wherein at least two polypeptide chains of the antibody construct are encoded within the same open reading frame, wherein consecutive polypeptide chains within said open reading frame are connected with a peptide linker comprising a 2A peptide.

The features and embodiments disclosed above for the nucleic acid product according to the first aspect also apply likewise to this nucleic acid product. In certain embodiments, the nucleic acid product of this aspect consists of one vector nucleic acid encoding an antibody construct consisting of a first and a second antibody heavy chain which are encoded within the same open reading frame connected to each other with a peptide linker comprising a 2A peptide.

### 2. HOST CELLS

In a second aspect, the present invention provides a host cell comprising the nucleic acid product as described herein.

The host cell may be of any cell type and in particular is a cell useful for recombinantly producing proteins. The host cell is in particular a cell capable of producing the antibody construct. In certain embodiments, the host cell in particular is a mammalian cell. The host cell may in particular be a rodent cell or a human cell. In certain embodiments, the mammalian cell is selected from, but not limited to, the group consisting of cells derived from mice, such as COP, L, C127, Sp2/0, NS0, NS1, At20 and NIH3T3; rats, such as PC12, PC12h, GH3, MtT, YB2/0 and Y0; hamsters, such as BHK, CHO and DHFR gene defective CHO; monkeys, such as COS1, COS3, COS7, CV1 and Vero; and humans, such as Hela, HEK293, CAP, retina-derived PER-C6, cells derived from diploid fibroblasts, myeloma cells and HepG2. In specific embodiments, the host cell is a Chinese hamster ovary (CHO) cell. The host cell may be suitable for suspension cultures and/or adherent cultures, and in particular can be used in suspension cultures.

In embodiments wherein the peptide linker comprises a protease recognition site, the host cell in particular expresses a protease which specifically recognizes and cleaves said protease recognition site. In particular, the peptide linker comprises a furin recognition site and the host cell expresses furin. The protease may intrinsically be expressed by the host cell or the host cell may be engineered to express the protease. Preferably, the protease is intrinsically expressed by the host cell.

In a fifth aspect, the present invention provides a method for producing a host cell according to the invention, comprising introducing the nucleic acid product as described herein into a host cell. The nucleic acid product is artificially introduced into the host cell. In particular, the nucleic acid product is introduced by transfection. Transfection in this respect may be transient or stable, and especially stable transfection is used. Hence, in certain embodiments the host cell comprises the nucleic acid product stably integrated into its genome.

The present invention further provides the use of the nucleic acid product for the transfection of a host cell. In particular, the host cell is a mammalian cell such as a Chinese hamster ovary (CHO) cell.

### 3. PRODUCTION METHOD

In a third aspect, the present invention provides a method for producing an antibody construct, comprising the steps of
(a) providing a host cell according to the second aspect of the present invention,
(b) cultivating the host cell in a cell culture under conditions which allow for production of the antibody construct,
(c) obtaining the antibody construct from the cell culture, and
(d) optionally processing the antibody construct.

In certain embodiments, the method further comprises between steps (a) and (b) the steps of
(a1) inoculating a cell culture medium with the host cell to provide a cell culture, and
(a2) cultivating the host cell in the cell culture under conditions which allow for increasing the number of cells in the cell culture.

Suitable conditions for cultivating the host cells, increasing their cell number and expressing the antibody construct depend on the specific host cell, vector and expression cassette used in the method. The skilled person can readily determine suitable conditions and they are also already known in the art for a plurality of host cells. In certain embodiments, nucleic acid product in the host cell comprises one or more selectable marker genes. In these embodiments, the culturing conditions in step (a2) and/or (b) may include the presence of corresponding selection agent(s) in the cell culture medium.

Obtaining the antibody construct from the cell culture in step (c) in particular includes isolating the antibody construct from the cell culture. Isolation of the antibody construct in particular refers to the separation of the antibody construct from the remaining components of the cell culture. The term “cell culture” as used herein in particular includes the cell culture medium and the cells. In certain embodiments, the antibody construct is secreted by the host cell. In these embodiments, the antibody construct is isolated from the cell culture medium. Separation of the antibody construct from the cell culture medium may be performed, for example, by chromatographic methods. Suitable methods and means for isolating the antibody construct are known in the art and can be readily applied by the skilled person.

The obtained antibody construct may optionally be subject to further processing steps such as e.g. further purification, modification and/or formulation steps in order to produce the antibody construct in the desired quality and composition. Such further processing steps and methods are generally known in the art. Suitable purification steps for example include affinity chromatography, size exclusion chromatography, anion- and/or cation exchange chromatography, hydrophilic interaction chromatography and reverse phase chromatography. Further steps may include virus inactivation, ultrafiltration and diafiltration. Formulation steps may include buffer exchange, addition of formulation components, pH adjustment, and concentration adjustment. Any combination of these and further steps may be used.

In certain embodiments, the method for producing an antibody construct further comprises as step (d) or part of step (d) the step of providing a pharmaceutical formulation comprising the antibody construct. Providing a pharmaceutical formulation comprising the antibody construct or formulating the antibody construct as a pharmaceutical composition in particular comprises exchanging the buffer solution or buffer solution components of the composition comprising the antibody construct. Furthermore, this step may include lyophilization of the antibody construct. In particular, the antibody construct is transferred into a composition only comprising pharmaceutically acceptable ingredients.

In certain embodiments, the polypeptide chains of the antibody product are produced more homogeneously compared to production of the polypeptide chains of the antibody construct using a nucleic acid product wherein each polypeptide chain of the antibody construct is encoded within a separate open reading frame. In certain embodiments, the relative amount of correctly assembled antibody constructs is higher compared to production of the antibody construct using a nucleic acid product wherein each polypeptide chain of the antibody construct is encoded within a separate open reading frame.

In a fourth aspect, the present invention further provides the use of the nucleic acid product as described herein or the host cell as described herein for the production of an antibody construct. The features and embodiments of the method for producing an antibody construct described herein likewise apply to this use.

### 4. SPECIFIC EMBODIMENTS

In the following, specific embodiments of the present invention are described. These embodiments can be combined with the further embodiments, features and examples described herein.

Embodiment 1. A nucleic acid product consisting of one or more vector nucleic acids encoding an antibody construct comprising at least three different polypeptide chains,
wherein the antibody construct comprises an antibody heavy chain; and
wherein at least two different polypeptide chains of the antibody construct are encoded within the same open reading frame,
wherein consecutive polypeptide chains encoded within said open reading frame are connected by a peptide linker comprising a 2A peptide.

Embodiment 2. A nucleic acid product consisting of one or more vector nucleic acids encoding an antibody construct comprising a first antibody heavy chain and a second antibody heavy chain which is different from the first heavy chain, wherein at least two polypeptide chains of the antibody construct are encoded within the same open reading frame, wherein consecutive polypeptide chains within said open reading frame are connected by a peptide linker comprising a 2A peptide.

Embodiment 3. The nucleic acid product according to embodiment 1 or 2, wherein the (first) antibody heavy chain is one of the at least two different polypeptide chains of the antibody construct which are encoded within the same open reading frame.

Embodiment 4. The nucleic acid product according to embodiment 3, wherein the N-terminal polypeptide chain encoded within said open reading frame is the (first) antibody heavy chain.

Embodiment 5. The nucleic acid product according to any one of embodiments 1 to 4, consisting of one vector nucleic acid encoding the antibody construct.

Embodiment 6. The nucleic acid product according to any one of embodiments 1 to 5, wherein the antibody construct comprises an antibody light chain binding to the heavy chain.

Embodiment 7. The nucleic acid product according to embodiment 6, wherein the at least two polypeptide chains encoded within the same open reading frame comprise the heavy chain and the light chain.

Embodiment 8. The nucleic acid product according to embodiment 7, wherein the light chain is the second polypeptide chain encoded within the open reading frame.

Embodiment 9. The nucleic acid product according to embodiment 8, wherein the peptide linker connects the C terminus of the heavy chain with the N terminus of the light chain.

Embodiment 10. The nucleic acid product according to any one of embodiments 6 to 9, wherein the heavy chain comprises a heavy chain variable region, and the light chain comprises a light chain variable region, wherein the heavy chain variable region and the light chain variable region form an antigen binding region.

Embodiment 11. The nucleic acid product according to any one of embodiments 1 to 10, wherein the antibody construct comprises a second antibody heavy chain.

Embodiment 12. The nucleic acid product according to embodiment 11, wherein the first heavy chain and the second heavy chain bind to each other using a knob-into-hole technology.

Embodiment 13. The nucleic acid product according to embodiment 11 or 12, wherein the at least two polypeptide chains encoded within the same open reading frame comprise the first heavy chain and the second heavy chain.

Embodiment 14. The nucleic acid product according to embodiment 11 or 12, wherein the second heavy chain is encoded within a second open reading frame different from the first open reading frame encoding the first heavy chain.

Embodiment 15. The nucleic acid product according to embodiment 14, wherein
at least two different polypeptide chains of the antibody construct are encoded within the second open reading frame,
the N-terminal polypeptide chain encoded within said second open reading frame is the second heavy chain which is connected to a further polypeptide chain encoded within said second open reading frame by a peptide linker comprising a 2A peptide.

Embodiment 16. The nucleic acid product according to any one of embodiments 11 to 15, wherein the antibody construct comprises a second light chain binding to the second heavy chain.

Embodiment 17. The nucleic acid product according to embodiment 16, wherein the second heavy chain and the second light chain are encoded within the same open reading frame comprising a peptide linker connecting these two polypeptide chains, wherein the peptide linker comprises a 2A peptide.

Embodiment 18. The nucleic acid product according to embodiment 17, wherein the peptide linker connects the C terminus of the second heavy chain with the N terminus of the second light chain.

Embodiment 19. The nucleic acid product according to any one of embodiments 16 to 18, wherein the second heavy chain comprises a heavy chain variable region, and the second light chain comprises a light chain variable region, wherein the chain variable regions of the second heavy chain and the second light chain form an antigen binding region.

Embodiment 20. The nucleic acid product according to any one of embodiments 1 to 19, wherein at least one, in particular each, heavy chain of the antibody construct comprises at least one heavy chain constant domain (CH), in particular at least CH2 or CH3, especially CH2 and CH3.

Embodiment 21. The nucleic acid product according to any one of embodiments 1 to 20, wherein at least one, in particular each, heavy chain of the antibody construct is capable of forming a homodimer and/or a heterodimer with another antibody heavy chain.

Embodiment 22. The nucleic acid product according to any one of embodiments 1 to 21, wherein at least one, in particular each, heavy chain of the antibody construct comprises a hinge region.

Embodiment 23. The nucleic acid product according to any one of embodiments 1 to 22, wherein at least one, in particular each, heavy chain of the antibody construct comprises a CH1 domain.

Embodiment 24. The nucleic acid product according to any one of embodiments 1 to 23, wherein at least one, in particular each, heavy chain of the antibody construct comprises a heavy chain variable domain (VH).

Embodiment 25. The nucleic acid product according to any one of embodiments 1 to 24, wherein at least one, in particular each, heavy chain of the antibody construct comprises one or more further polypeptide moieties

Embodiment 26. The nucleic acid product according to any one of embodiments 1 to 25, wherein at least one, in particular each, heavy chain of the antibody construct comprises an antigen binding region.

Embodiment 27. The nucleic acid product according to any one of embodiments 1 to 26, wherein at least one, in particular each, light chain of the antibody construct comprises a light chain constant domain (CL).

Embodiment 28. The nucleic acid product according to any one of embodiments 1 to 27, wherein at least one, in particular each, light chain of the antibody construct is capable of forming a heterodimer with an antibody heavy chain.

Embodiment 29. The nucleic acid product according to any one of embodiments 1 to 28, wherein at least one, in particular each, light chain of the antibody construct comprises a light chain variable domain (VL).

Embodiment 30. The nucleic acid product according to any one of embodiments 1 to 29, wherein at least one, in particular each, light chain of the antibody construct comprises one or more further polypeptide moieties Embodiment 31. The nucleic acid product according to any one of embodiments 1 to 30, wherein at least one, in particular each, light chain of the antibody construct comprises an antigen binding region.

Embodiment 32. The nucleic acid product according to any one of embodiments 1 to 31, wherein the first heavy chain of the antibody construct is encoded with a signal peptide at the N terminus.

Embodiment 33. The nucleic acid product according to embodiment 32, wherein each heavy chain of the antibody construct is encoded with a signal peptide at the N terminus.

Embodiment 34. The nucleic acid product according to any one of embodiments 1 to 33, wherein the first light chain of the antibody construct is encoded with a signal peptide at the N terminus.

Embodiment 35. The nucleic acid product according to embodiment 34, wherein each light chain of the antibody construct is encoded with a signal peptide at the N terminus.

Embodiment 36. The nucleic acid product according to any one of embodiments 1 to 35, wherein one or more vector nucleic acids encode within a first open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide; and
(v) a first light chain comprising a first light chain variable region; and
wherein the one or more vector nucleic acids encode within a second open reading frame, in the direction from N terminus to C terminus,
(i) a signal peptide;
(ii) a second heavy chain comprising a second heavy chain variable region;
(iii) a linker peptide comprising a 2A peptide;
(iv) optionally a signal peptide; and
(v) a second light chain comprising a second light chain variable region;
wherein the first heavy chain variable region and the first light chain variable region form an antigen binding region capable of binding a first antigen; and
wherein the second heavy chain variable region and the second light chain variable region form an antigen binding region capable of binding a second antigen; and
wherein the first heavy chain and the second heavy chain bind to each other using a knob-into-hole technology.

Embodiment 37. The nucleic acid product according to any one of embodiments 1 to 35, wherein the vector nucleic acid encodes within the same open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide;
(v) a first light chain comprising a first light chain variable region;
(vi) a linker peptide comprising a 2A peptide;
(vii) optionally a signal peptide;
(viii) a second heavy chain comprising a second heavy chain variable region;
(ix) a linker peptide comprising a 2A peptide;
(x) optionally a signal peptide; and
(xi) a second light chain comprising a second light chain variable region;
wherein the first heavy chain variable region and the first light chain variable region form an antigen binding region capable of binding a first antigen; and
wherein the second heavy chain variable region and the second light chain variable region form an antigen binding region capable of binding a second antigen; and
wherein the first heavy chain and the second heavy chain bind to each other using a knob-into-hole technology.

Embodiment 38. The nucleic acid product according to any one of embodiments 1 to 35, wherein the one or more vector nucleic acids encode within a first open reading frame, in the direction from N terminus to C terminus,
(i) a signal peptide;
(ii) a first heavy chain comprising a heavy chain variable region;
(iii) a linker peptide comprising a 2A peptide;
(iv) optionally a signal peptide; and
(v) a light chain comprising a light chain variable region; and
wherein the one or more vector nucleic acids encode within a second open reading frame, in the direction from N terminus to C terminus,
(i) a signal peptide; and
(ii) a second heavy chain; and
wherein the heavy chain variable region of the first heavy chain and the light chain variable region form an antigen binding region capable of binding a first antigen; and
wherein the second heavy chain comprises an antigen binding region capable of binding a second antigen; and
wherein the first heavy chain and the second heavy chain bind to each other using a knob-into-hole technology.

Embodiment 39. The nucleic acid product according to any one of embodiments 1 to 35, wherein the one or more vector nucleic acids encode within a first open reading frame, in the direction from N terminus to C terminus,
(i) a signal peptide;
(ii) a first heavy chain;
(iii) a linker peptide comprising a 2A peptide;
(iv) optionally a signal peptide; and
(v) a second heavy chain; and
wherein the one or more vector nucleic acids encode within a second open reading frame, in the direction from N terminus to C terminus,
(i) a signal peptide; and
(v) a light chain comprising a light chain variable region; and
wherein
(a) the first heavy chain comprises a heavy chain variable region which forms an antigen binding region with the light chain variable region, capable of binding a first antigen; and the second heavy chain comprises an antigen binding region capable of binding a second antigen; or
(b) the first heavy chain comprises an antigen binding region capable of binding a first antigen; and the second heavy chain comprises a heavy chain variable region which forms an antigen binding region with the light chain variable region, capable of binding a second antigen; and
wherein the first heavy chain and the second heavy chain bind to each other using a knob-into-hole technology.

Embodiment 40. The nucleic acid product according to any one of embodiments 1 to 35, wherein the vector nucleic acid encodes within the open reading frame, in the direction from N terminus to C terminus,
(i) a signal peptide;
(ii) a first heavy chain;
(iii) a linker peptide comprising a 2A peptide;
(iv) optionally a signal peptide;
(v) a second heavy chain comprising a heavy chain variable region;
(vi) a linker peptide comprising a 2A peptide;
(vii) optionally a signal peptide; and
(viii) a light chain comprising a light chain variable region; and
wherein the first heavy chain comprises an antigen binding region capable of binding a first antigen; and
wherein the heavy chain variable region of the second heavy chain and the light chain variable region form an antigen binding region capable of binding a second antigen; and
wherein the first heavy chain and the second heavy chain bind to each other using a knob-into-hole technology.

Embodiment 41. The nucleic acid product according to any one of embodiments 1 to 40, wherein the 2A peptide is derived from a virus selected from the group consisting of foot-and-mouth disease virus, equine rhinitis A virus, porcine teschovirus-1, and Thosea asigna virus.

Embodiment 42. The nucleic acid product according to any one of embodiments 1 to 41, wherein the peptide linker(s) further comprises a protease recognition site N terminal of the 2A peptide.

Embodiment 43. The nucleic acid product according to embodiment 42, wherein the protease recognition site is a furin recognition site.

Embodiment 44. The nucleic acid product according to any one of embodiments 1 to 43, wherein the peptide linker(s) comprise an amino acid sequence according to SEQ ID NO: 25.

Embodiment 45. The nucleic acid product according to any one of embodiments 1 to 44, providing for a more homogeneous cellular production of the polypeptide chains of the antibody construct compared to production of the polypeptide chains of the antibody construct using a nucleic acid product wherein each polypeptide chain of the antibody construct is encoded within a separate open reading frame.

Embodiment 46. The nucleic acid product according to embodiment 45, wherein the amounts of the mRNAs coding for the different polypeptide chains of said antibody construct in the cell do not differ by more than factor 20.

Embodiment 47. The nucleic acid product according to embodiment 45, wherein the amounts of the mRNAs coding for the different polypeptide chains of said antibody construct in the cell do not differ by more than factor 10.

Embodiment 48. The nucleic acid product according to embodiment 45, wherein the amounts of the mRNAs coding for the different polypeptide chains of said antibody construct in the cell do not differ by more than factor 8.

Embodiment 49. The nucleic acid product according to embodiment 45, wherein the amounts of the mRNAs coding for the different polypeptide chains of said antibody construct in the cell do not differ by more than factor 5.

Embodiment 50. The nucleic acid product according to any one of embodiments 1 to 49, providing for a higher relative amount of correctly assembled antibody constructs after expression of the polypeptide chains of the antibody construct compared to the relative amount of correctly assembled antibody constructs after expression of the polypeptide chains of the antibody construct using a nucleic acid product wherein each polypeptide chain of the antibody construct is encoded within a separate open reading frame.

Embodiment 51. The nucleic acid product according to embodiment 50, wherein the relative amount of correctly assembled antibody constructs after expression of the polypeptide chains of the antibody construct is at least 5 percentage points higher than the relative amount of correctly assembled antibody constructs using a nucleic acid product wherein each polypeptide chain is encoded within a separate open reading frame.

Embodiment 52. The nucleic acid product according to embodiment 50, wherein the relative amount of correctly assembled antibody constructs after expression of the polypeptide chains of the antibody construct is at least 10 percentage points higher than the relative amount of correctly assembled antibody constructs using a nucleic acid product wherein each polypeptide chain is encoded within a separate open reading frame.

Embodiment 53. The nucleic acid product according to embodiment 50, wherein the relative amount of correctly assembled antibody constructs after expression of the polypeptide chains of the antibody construct is at least 15 percentage points higher than the relative amount of correctly assembled antibody constructs using a nucleic acid product wherein each polypeptide chain is encoded within a separate open reading frame.

Embodiment 54. The nucleic acid product according to embodiment 50, wherein the relative amount of correctly assembled antibody constructs after expression of the polypeptide chains of the antibody construct is at least 20 percentage points higher than the relative amount of correctly assembled antibody constructs using a nucleic acid product wherein each polypeptide chain is encoded within a separate open reading frame.

Embodiment 55. The nucleic acid product according to any one of embodiments 1 to 54, wherein the open reading frame coding for two or more polypeptide chains of the antibody construct is part of an expression cassette which enables expression of the open reading frame.

Embodiment 56. The nucleic acid product according to any one of embodiments 1 to 55, wherein the vector nucleic acids are plasmids.

Embodiment 57. A host cell comprising the nucleic acid product according to any one of embodiments 1 to 56.

Embodiment 58. The host cell according to embodiment 57, being a mammalian cell, especially a human or rodent cell, for example a CHO cell.

Embodiment 59. A method for producing an antibody construct, comprising the steps of
(a) providing a host cell according to embodiment 57 or 58,
(b) cultivating the host cell in a cell culture under conditions which allow for production of the antibody construct,
(c) obtaining the antibody construct from the cell culture, and
(d) optionally processing the antibody construct.

Embodiment 60. The method according to embodiment 59, wherein step (c) comprises isolating the antibody construct and/or separating the antibody construct from the remaining components of the cell culture.

Embodiment 61. The method according to embodiment 59 or 60, wherein step (d) comprises formulating the antibody construct as a pharmaceutical composition.

Embodiment 62. The method according to any one of embodiments 59 to 61, wherein the polypeptide chains of the antibody product are produced more homogeneously compared to production of the polypeptide chains of the antibody construct using a nucleic acid product wherein each polypeptide chain of the antibody construct is encoded within a separate open reading frame.

Embodiment 63. The method according to embodiment 62, wherein the amounts of the mRNAs coding for the different polypeptide chains of said antibody construct in the cell do not differ by more than factor 20.

Embodiment 64. The method according to embodiment 62, wherein the amounts of the mRNAs coding for the different polypeptide chains of said antibody construct in the cell do not differ by more than factor 10.

Embodiment 65. The method according to embodiment 62, wherein the amounts of the mRNAs coding for the different polypeptide chains of said antibody construct in the cell do not differ by more than factor 8.

Embodiment 66. The method according to embodiment 62, wherein the amounts of the mRNAs coding for the different polypeptide chains of said antibody construct in the cell do not differ by more than factor 5.

Embodiment 67. The method according to any one of embodiments 51 to 54, wherein the relative amount of correctly assembled antibody constructs is higher compared to production of the antibody construct using a nucleic acid product wherein each polypeptide chain of the antibody construct is encoded within a separate open reading frame.

Embodiment 68. The method according to embodiment 67, wherein the relative amount of correctly assembled antibody constructs is at least 5 percentage points higher than the relative amount of correctly assembled antibody constructs using a nucleic acid product wherein each polypeptide chain is encoded within a separate open reading frame.

Embodiment 69. The method according to embodiment 67, wherein the relative amount of correctly assembled antibody constructs is at least 10 percentage points higher than the relative amount of correctly assembled antibody constructs using a nucleic acid product wherein each polypeptide chain is encoded within a separate open reading frame.

Embodiment 70. The method according to embodiment 67, wherein the relative amount of correctly assembled antibody constructs is at least 15 percentage points higher than the relative amount of correctly assembled antibody constructs using a nucleic acid product wherein each polypeptide chain is encoded within a separate open reading frame.

Embodiment 71. The method according to embodiment 67, wherein the relative amount of correctly assembled antibody constructs is at least 20 percentage points higher than the relative amount of correctly assembled antibody constructs using a nucleic acid product wherein each polypeptide chain is encoded within a separate open reading frame.

Embodiment 72. Use of the nucleic acid product according to any one of embodiments 1 to 56 or the host cell according to embodiment 57 or 58 for the production of an antibody construct.

Embodiment 73. A method for producing a host cell according to embodiment 57 or 58, comprising introducing the nucleic acid product according to any one of embodiments 1 to 56 into a host cell.

### FIGURES

FIG. 1 shows the principle of a standard two-vector setup for expression of a bispecific antibody construct (bsAb) and the structure of the bsAb using knob-into-hole (KiH) technology.

FIG. 2 shows the principle of cellular mechanisms occurring if using a 2A/furin linker peptide with a vector setup including two plasmids and two mRNAs combining two chains in one expression cassette.

FIG. 3 shows an overview of the molecule structure as well as standard and 2A/furin vector setups tested for expression of a bispecific antibody.

FIG. 4 shows relative mRNA levels of the heavy and light chains using the different vector setups illustrated in FIG. 3.

FIG. 5 shows pool productivities of the bispecific antibody using the different vector setups illustrated in FIG. 3.

FIG. 6 shows 2A/furin vector setups tested for expression of a bispecific antibody.

FIG. 7 shows relative mRNA levels of the heavy and light chains using the different vector setups illustrated in FIG. 6.

FIG. 8 shows pool productivities of the bispecific antibody using the different vector setups illustrated in FIG. 6.

FIG. 9 shows standard (B) and furin/2A (C) vector setups of a trifunctional antibody construct (A).

FIG. 10 shows pool productivities and quality analyses by SEC and labchip of the trifunctional antibody construct using the standard (A) and furin/2A (B) vector setups illustrated in FIG. 9. Shown are the antibody titers and the percentages of the main peak obtained by SEC and labchip analysis, respectively.

### EXAMPLES

### Example 1: Standard Vector Design and Vector Design According to the Invention

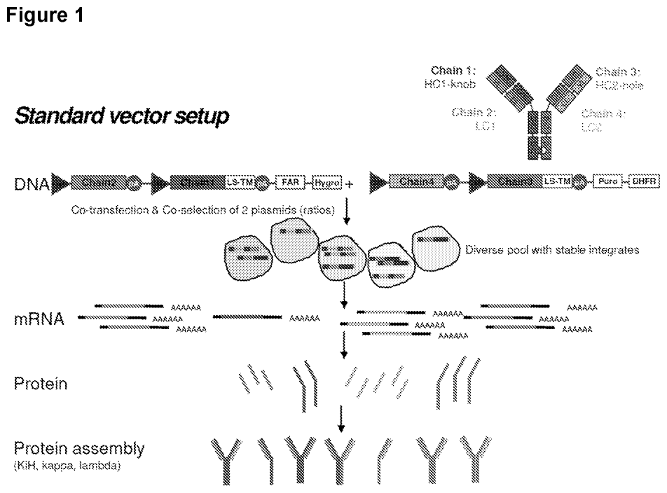

In comparison to monoclonal antibodies, complexity to develop cell lines that express correctly assembled bispecific antibody constructs (bsAbs) is increased because four chains need to be expressed. Co-transfection and co-selection of two separate plasmids, each expressing one of the two light chains (LCs) and heavy chains (HCs) in separate expression cassettes, might lead to inhomogeneous pools of cells having integrated none, one or two plasmids or an uneven number of the two plasmids. In addition, different transcription and translation efficiencies of the individual chains might lead to imbalanced mRNA and protein levels of the individual chains. Inhomogeneous distribution of protein chains disturbs correct protein assembly and an access of individual chains leads to unwanted species, e.g. mispaired antibody constructs, half-assembled bispecific antibodies, and homodimers, complicating the purification process. The standard vector setup and production process is illustrated in FIG. 1.

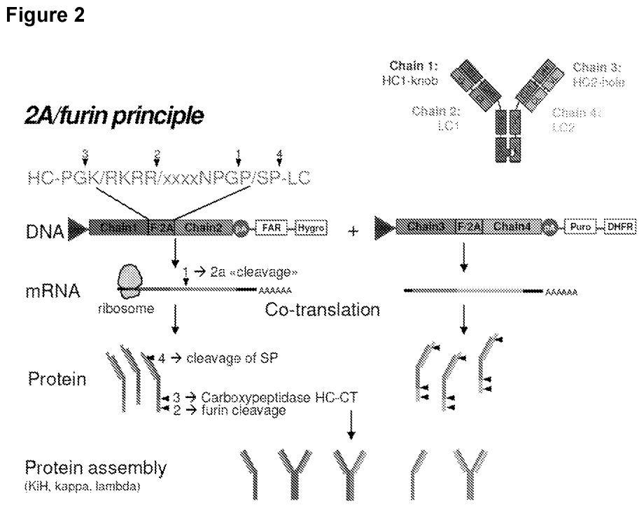

In the newly developed vector setup, the heavy and light chains of one arm of the bispecific antibody are encoded within one open reading frame, separated by a linker peptide comprising a furin recognition site and a 2A peptide. Transcription leads to one mRNA encoding chain1-2AF-chain2 (HC1-knob-furin/2A linker-LC1) from one plasmid and to another mRNA encoding chain3-2AF-chain4 (HC2-hole-furin/2A linker-LC2) from the second plasmid (see FIG. 2). Two distinct proteins are obtained after translation of the 2A peptide by cleaving of the 2A peptide (1). Co-translation of the corresponding light and heavy chain from one mRNA is advantageous for correct chain pairing and protein assembly. Furin (2) cleaves the remaining amino acids of the N terminal 2A peptide in the Golgi apparatus. Carboxypeptidase D (3) cleaves the C terminal lysine of the heavy chain as well as the remaining furin cleavage site and signal peptide peptidases (4) cleave the remaining C terminal proline of the 2A peptide together with the signal peptide of the light chain.

As demonstrated in the following examples, the inventive vector design is superior
1) Combining expression of individual chains from one mRNA leads to balanced mRNA and protein levels and overcomes expression imbalances.
2) Pool productivities are comparable.
3) The percentage of correct paired bispecific antibody constructs increases if LC1 and HCl are encoded on one single mRNA and LC2 and HC2 on a second single mRNA as well as if all 4 chains are encoded on 1 single mRNA.
4) The N- and C-termini of the heavy chains and light chains are correctly processed if the 2 proteins are expressed in the order HC-furin/2A-LC. If the furin/2A peptide is at the C-terminus of the light chain, different amino acid extensions remaining from the furin recognition site were observed (R, RK and RKR).
5) Percentage of mispaired species for the bispecific antibody constructs with knob-into-hole technology (KiH) are reduced.

### Example 2: Comparison Between Standard and Different Furin/2A Vector Designs

A single vector can encode several chains allowing transfection and selection of one plasmid instead of co-transfection of two separate plasmids. Transfection and selection of a single plasmid increases the probability to obtain a more homogenous pool: cells either have integrated the plasmid or not. Co-transfection and co-selection might lead to more inhomogeneous pools: cells have integrated none, one or two plasmids or integrate an uneven number of the two plasmids.

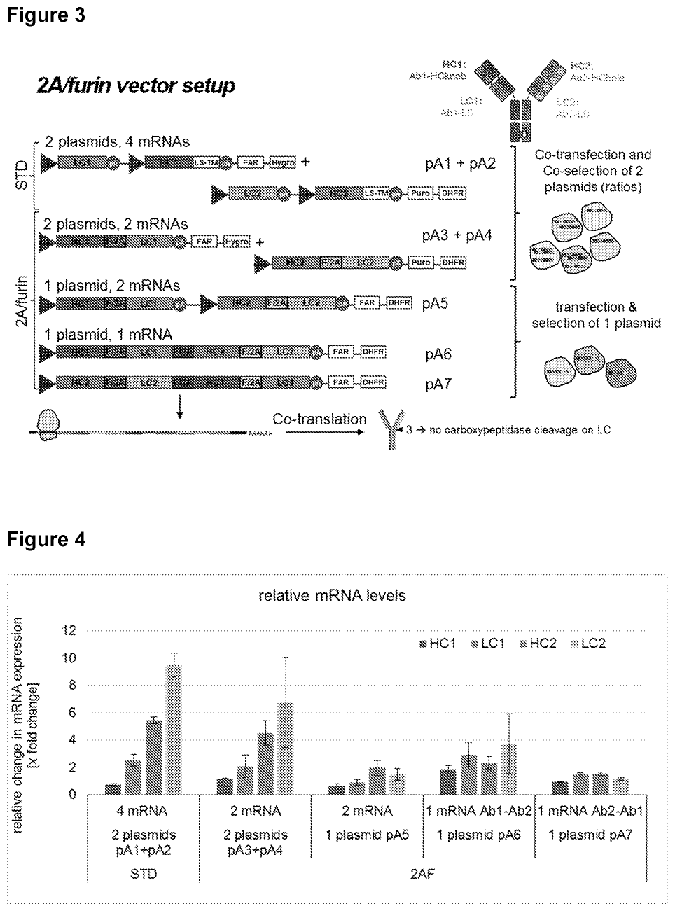

Four different furin/2A vector designs were compared to the standard vector design. According to the standard design, each of the four different polypeptide chains of the bispecific antibody construct is encoded in a separate open reading frame, leading to four separate mRNAs. The expression cassettes of the heavy and light chain pair of each arm of the bispecific antibody were present on a single plasmid so that the host cell was transfected with two plasmids (“2 plasmids, 4 mRNAs”; see FIG. 3, “STD”).

In two of the four furin/2A vector designs, each heavy and light chain pair was encoded in one open reading frame, coding first for the heavy chain, then for the furin/2A linker, and then for the light chain. These two open reading frames were either present on separate plasmids (“2 plasmids, 2 mRNAs”) or on the same plasmid (“1 plasmid, 2 mRNAs”). In the other two furin/2A vector designs, all polypeptide chains of the bispecific antibody are encoded within the same open reading frame in the order HCl-furin/2A-LC1-furin/2A-HC2-furin/2A-LC2 (“1 plasmid, 1 mRNA”; see FIG. 3, “2A/furin”). In the two different setups, the coding regions of the two arms of the bispecific antibody were switched.

The respective plasmids were transferred into CHO cells for production of the bispecific antibody. Expression of the mRNAs and production of correctly assembled and mispaired bispecific antibody constructs were determined

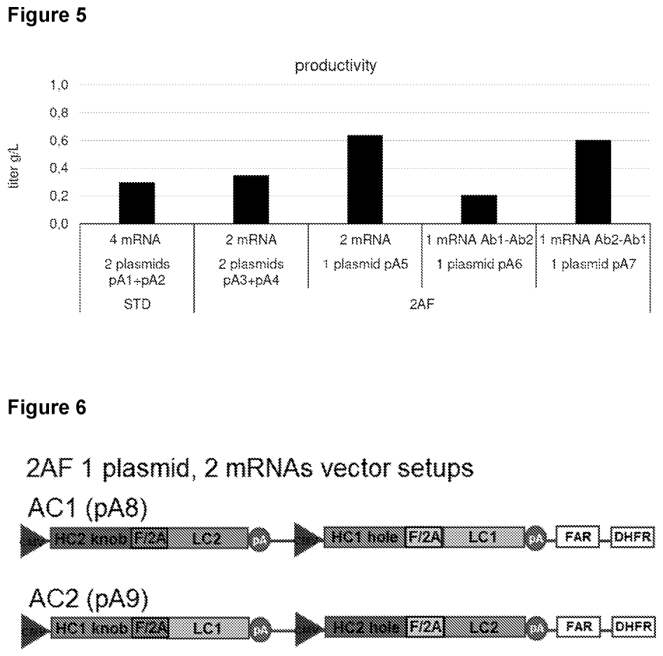

The results in FIG. 4 demonstrate that mRNA levels of the different polypeptide chains were highly imbalanced for the standard vector setup. The “2 plasmids, 2 mRNAs setup” improved the balance while the setups with only 1 plasmid showed even more balanced mRNA levels. The productivity was also markedly increased for the 1 plasmid setups (see FIG. 5). In the 1 plasmid, 1 mRNA vector design, the order in which the different arms of the bispecific antibody are encoded within the open reading frame may have an influence on the productivity.

The correct assembly of the bispecific antibody construct and the formation of mispaired constructs, homodimers and half antibodies are summarized in Table 1:

TABLE 1

| | | | Intact/deglyco MS analysis | | | | | |
|---|---|---|---|---|---|---|---|---|
| | | | | Mispaired | homodimer | | | |
| | | | Correct** | L1H1L1H2 | L2H2L2H2 | Half mAb | | |
| | | | bispecific (%) | (%) | (%) | L2H2 (%) | Other | LC- |
| | | | [symbol] or [symbol] | [symbol] | [symbol] | [symbol] | (%) | Extensions |
| STD | 2 plasmids, | pA1 | 28* | n.d. | 58* | 8* | 6* | n.d. |
| | 4 mRNAs | pA2 | | | | | | |
| 2AF | 2 plasmids, | pA3 | 44* | n.d. | 34* | 6* | 17* | n.d. |
| | 2 mRNAs | pA4 | | | | | | |
| | 1 plasmid, | pA5 | 49* | n.d. | 27* | 14* | 10* | n.d. |
| | 2 mRNAs | | | | | | | |
| | 1 plasmid, | pA6 | 100* | n.d. | n.d. | n.d. | n.d. | +RKR |
| | 1 mRNA (Ab1-Ab2) | | | | | | | |
| | 1 plasmid, | pA7 | 77* | 7* | n.d. | n.d. | 16* | +RK |
| | 1 mRNA (Ab2-Ab1) | | | | | | | +R |

*Semi-quantification only as complete separation (UV) of all species cannot be achieved.
All values (%) are calculated by combining UV areas and MS intensities.
**correct bispecific by intact LC-MS may also comprise complete LC swapped species

Table 1 shows mass spectrometry data of protein A captured material for standard vector design (“STD”) and different furin/2A vector setups (“2AF”). Percentage of correctly assembled bispecific antibodies was increased for all furin/2A vector setups in comparison to the standard vector setup. Highest percentage of correctly assembled bispecific antibody was detected for furin/2A vector setups encoding all 4 chains on one mRNA and vector. However, light chain extensions for the light chain with the furin/2A peptide at the C-terminus were observed.

### Example 3: Evaluation of Production of a Bispecific Antibody Construct Using Various Furin/2A Vector Setups with 2 mRNAs on 1 Plasmid

A bispecific antibody construct was produced with the standard vector setup and with the “1 plasmid, 2 mRNAs” furin/2A vector setup, using different designs. Each arm of the bispecific antibody was encoded in one open reading frame with the heavy chain at the N terminus, followed by a furin/2A linker peptide and the light chain. Two furin/2A vector setups were designed, one with the open reading frame coding for the first arm located 5′ of the open reading frame coding for the second arm and one in reversed order.

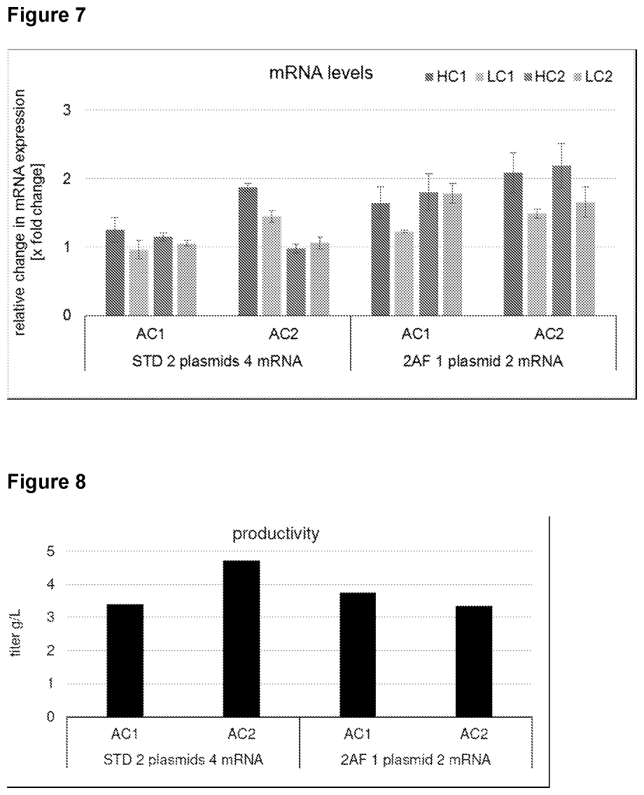

mRNA levels of the different polypeptide chains were highly imbalanced for some of the standard vector setups (see FIG. 7, “STD 2 plasmids 4 mRNA”). In contrast, for the furin/2A vector setups, the mRNA levels were well balanced. The productivity of all vector setups was comparable (see FIG. 8).

The correct assembly of the bispecific antibody construct and the formation of mispaired constructs, homodimers and half antibodies are summarized in Table 2:

TABLE 2

| | | Intact/deglyco MS analysis | | | | |
|---|---|---|---|---|---|---|
| | | Correct** | Mispaired | homodimer | | |
| | | bispecific (%) | L1H1L1H2 (%) | L1H1L1H1 (%) | Half mAb | Other |
| | | [symbol] or [symbol] | [symbol] | [symbol] | (%) | (%) |
| STD | AC1 | 52.4* | 31.4* | 4.9* | 9.7* (L2H1) | 1.6* |
| 2 plasmid, | AC2 | 33.2* | 33.1* | n.d. | 30.9* (L2H1 | 2.8* |
| 4 mRNA | | | | | and L1H1) | |
| 2AF | AC1 | 81.4* | 7.1* | n.d. | 10.1* (L2H1) | 1.4* |
| 1 plasmid, | AC2 | 87.1* | 9.2* | n.d. | 1.1* (L1H1) | 2.6* |
| 2 mRNA | | | | | | |

*Semi-quantification only as complete separation (UV) of all species cannot be achieved.
All values (%) are calculated by combining UV areas and MS intensities.
**correct bispecific by intact LC-MS may also comprise complete LC swapped species

Table 2 shows mass spectrometry data of protein A captured material comparing different standard vector setups (“STD”) and furin/2A vector setups (“2AF”). Expression of furin/2AF “1 plasmid, 2 mRNAs” vector setups increased the percentage of correctly assembled bispecific antibodies and decreased the percentage of the mispaired species for the tested candidates. The results also show that correct light chain pairing is achieved in the furin/2A vector setups as these setups produced less than 10% mispaired light chains compared to more than 30% for the standard setups. The reason for this putatively is the production of the heavy chain-light chain pair in close proximity inside the cell since they are encoded by the same mRNA.

Low percentage of mispairing decreases efforts needed for the purification processes and minimizes the possibility of the completely swapped light chain species, which cannot be removed in a purification process.

### Example 4: Evaluation of Production of Another Antibody Construct Using Different Furin/2A Vector Setups

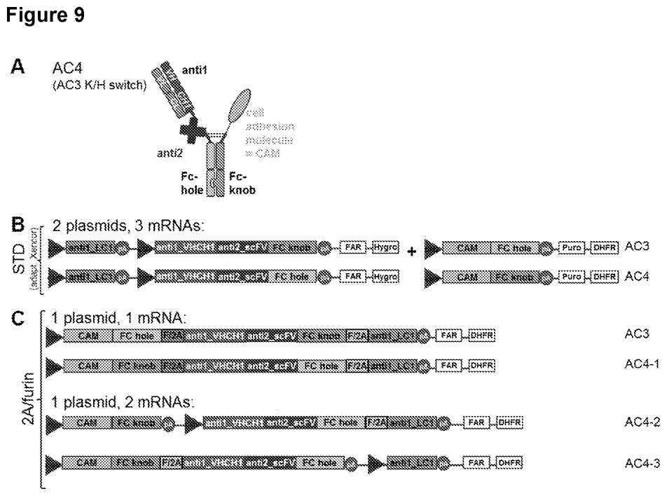

Non-standard antibody constructs were also tested for expression with the new furin/2A vector setup. An exemplary antibody construct comprised one arm with a heavy chain and a light chain forming a normal antigen binding region specific for a first antigen (anti1), wherein a scFv fragment against a second antigen (anti2) was fused between CH1 and hinge region of the heavy chain. The second arm was a heavy chain constant region comprising hinge, CH2 and CH3 which attached to the first arm using knob-into-hole technology and wherein a cell adhesion molecule is fused to the N terminus of the hinge region (see FIG. 9A). Two of these constructs were designed, differing in the knob and hole mutations of the CH3 domains (antibody construct AC3: arm 1-knob/arm 2-hole, and antibody construct AC4: arm 1-hole/arm 2-knob).

According to the standard vector setup, one plasmid comprising expression cassettes for the light chain and the heavy chain of the first arm and one plasmid comprising an expression cassette for the second arm were used (see FIG. 9B). In the furin/2A vector setups, only one plasmid with either one expression cassette or two expression cassettes was used. In the design with only one open reading frame, the coding regions were ordered from 5′ to 3′ as HC2-furin/2A-HC1-furin/2A-LC1. In the design with two open reading frames, the furin/2A linker was either between HC2 and HC1 or between HC1 and LC1 (see FIG. 9C).

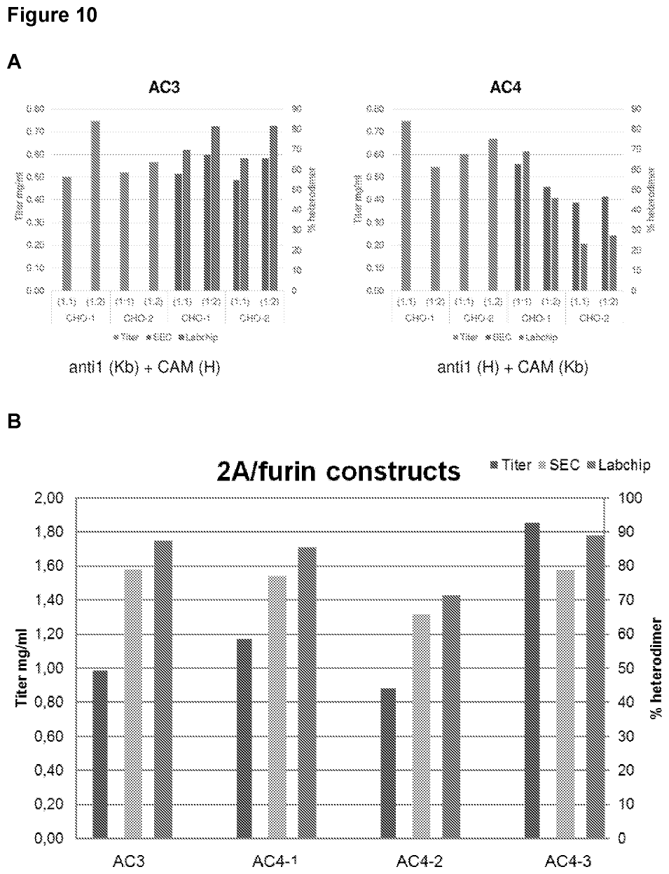

Different CHO cell lines were transfected with the plasmids and the antibody construct was produced under standard conditions. For the standard vector setup, the two plasmids were transfected in 1:1 ratio and in 1:2 ratio. Pool productivities of the standard vector setups were between 0.5 and 0.75 g/L for both candidates depending on the used ratio of the plasmids during transfection and host cell line used. Quality was analyzed by SEC and labchip and the percentage of the main peak is shown (see FIG. 10A).

FIG. 10B shows that pool productivities of the furin/2A vector setups were increased in comparison to pools generated with the standard approach. Quality was analyzed by SEC and labchip. The percentage of the main peak increased to 79% (SEC) and 87% (Labchip) for the first knob-into-hole design and up to 79% (SEC) and 89% (Labchip) for the second knob-into-hole design using the furin/2A vector setup in comparison to the standard approach.

These data demonstrate that productivity and correct assembly of the antibody construct improved using the furin/2A vector design.

### Example 5: Materials and Methods

The following materials and methods were used in Examples 1 to 4.

1. Expression Vector Construction The vectors used in the examples consist of following elements: hCMV promoter/enhancer driving expression of the individual genes needed for assembly of the antibody constructs, polyadenylation signal (polyA), folic acid receptor and DHFR gene as selection markers, E. coli origin (ColE on) of replication and the beta-lactamase gene for ampicillin (amp) resistance to enable amplification in bacteria. Different plasmid setups were evaluated and more details are provided within the figures.

### 2. Cell Lines, Cultivation, Transfection and Selection

Two different parental CHO cell lines were used as host cell lines for the production of the antibody constructs. Host cell lines were derived from the CHO-K1 cell line. A single vial from the CHO line was used to prepare the recombinant cell lines. CHO cell lines were cultivated in shake flasks in a non-humidified shaker cabinet at 150 rpm, 10% CO₂ at 36.5° C. in suspension in proprietary, chemically defined culture media. Cell viabilities and growth rates were monitored by means of an automated system (ViCell, Beckman Coulter). Cells were passaged 2-3 times per week into fresh medium and were maintained in logarithmic growth phase.

SwaI linearized expression plasmids encoding the antibody constructs were transfected by electroporation (Amaxa Nucleofection system, Lonza, Germany). The transfection reaction was performed in chemically defined cultivation medium, according to the manufactures instructions. The parental CHO cells used for transfection were in exponential growth phase with cell viabilities higher than 95%. Transfections were performed with 5×10⁶ cells per transfection. Immediately, after transfection cells were transferred into shake flasks, containing chemically defined cultivation medium. Cell pools were incubated for 48 hours at 36.5° C. and 10% CO2 before starting the selection process.

A selection procedure was carried out using the selection markers encoded by the individual expression vectors, as described above. Both proteins (FoIR and DHFR) are participating in the same molecular pathway; the FoIR is transporting folic acid as well as the folate analogue MTX into the cell, the DHFR is converting it into vital precursors for purine and methionine synthesis. Combining them as selective principle, a particular strong selective regime can be taken to enrich for recombinant cells expressing both recombinant protein.

48 h after transfection and growth under low folate conditions, additional selective pressure was applied by adding 10 nM MTX to the chemically defined cultivation medium. After pool recovery cells were frozen in culture medium, supplemented with 7.5% DMSO and cell pellets prepared.

### 3. Gene Expression Analysis by Quantitative Real-Time PCR

RNA extraction was performed using the Qiagen RNeasy Mini Kit according to the manufactures instructions. For real-time qPCR, cDNA was synthesized from 200 ng/μl diluted RNA using the High Capacity RNA-to-cDNA Master Mix (Applied Biosystems) and 10× diluted cDNAs were analyzed in triplicates using the QuantiFast SYBR Green PCR Kit (Qiagen). As endogenous control for normalization GAPDH was amplified. Amplification and analysis was performed using the ABI PRISM® 7900HT Sequence Detection System. For calculation of relative quantities (RQ) of gene expression for sample comparison the comparative 2^{-ΔΔCt} method was used and the data normalized.

### 4. Upstream Processing

Subsequent to selection, material was produced either in shake flask fed batch cultures or tube spin bioreactors. Fed batch cultures were inoculated with a cell seeding density of 4E5 vc/ml (addition of proprietary feed solutions starting on day 3 and cultivation temperature shift to 33° C. on day 5). During the cultivation in-process controls were performed to monitor the concentration of the antibody construct. The individual culture was cultivated over a period of 14 days. At the end of the cultivation process cells were separated from the culture supernatant by centrifugation followed by sterile filtration before further downstream processing. Volumetric productivities of selected pools were determined by Protein A HPLC in cell culture supernatants to determine all kind of product and related impurities carrying a Fc part.

### 5. Capturing by Affinity Liquid Chromatography on MabSelect™ SuRe™

Antibody construct and potential antibody construct variants carrying an Fc-part were captured from cell-free supernatant by an affinity liquid chromatography (ALC) step on MabSelect™ SuRe™.

Chromatography was performed at room temperature. Columns were equilibrated with 20 mM Na₂HPO₄/NaH₂PO₄, pH 7.0 before loading. To deplete unspecific bound impurities from product (and product variants), such as host cell proteins (HCP), media components and DNA the chromatography column was washed with 250 mM Arginine-HCl, 1 M NaCl, 88 mM NaOH, pH 9.0 followed by equilibration buffer after loading cell-free supernatant onto the ALC column. Antibody construct and potential antibody construct variants were eluted from the chromatographic column by using 50 mM acetic acid, pH 3.0. The pH of the ALC eluates were adjusted to ˜pH 5.0 with 0.1 or 1 M Tris or 0.5 M Bis-Tris prior to storage at 2-8° C. respectively for analytical assessment.

### 6. Analytical Characterization and Purity Assessment

Protein A captured material from pools was carefully evaluated by different analytical methodologies to judge product characteristics and quality parameters.

### (a) LC-MS Screening of Intact Antibody Construct and Variants

100 μg of protein A purified antibody construct samples were lyophilized in a 96-well plate and resolved in 100 ul of 50 nM Tris-HCl pH 7.5) buffer. Samples were de-glycosylated by PNGaseF (New England Biolabs) at 37° C. for 18 hours. Samples were measured by LC-ESI-MS on an UPLC (Waters) connected to a Vion Q-TOF mass spectrometer (Waters), using a MassPREP Micro Desalting column 2 1×5 mm (Waters) at 80° C. A linear gradient was applied at 0.3 ml/min with mobile phase A: 0.1 formic acid in water, mobile phase B: 0.1% FA in acetonitrile: 0-2 min 5% B, 2-12 min 5-90% B. MS parameters: ESI+Resolution mode, Capillary voltage 3 kV, sampling cone 40 V, source temperature 150° C., de-solvation temperature 400° C. Data was processed by automatic MaxEnti deconvolution with Genedata MS refiner software. Identification and relative quantification of antibody construct species and mispaired variants is based on the match to the theoretical mass and the corresponding relative peak intensity of the de-convoluted mass spectrum.

### (b) Labchip

Non-Reducing Labchip runs were carried using HT Protein Express Reagent Kit and HT Protein Express LabChip from Caliper LifeSciences according to manufacturer's instructions. Protein A purified samples were prepared by adding 35 uL IAM Buffer (300 uL of 250 mM IAM in 3 mL Protein Express sample buffer) to 5 uL sample (1 mg/ml). The samples were heated to 70° C. for 10 mins and then added 70 uL of water before the Labchip run on LabChip GXII from Caliper LifeSciences.

### (c) Size Exclusion Chromatography

Protein A purified samples were subjected to Waters UPLC BEH200 (Waters #186005225, 1.7 um, 4.6 mm×150 mm) SEC column, pore size 200 A. Mobile phase was 50 mM sodium phosphate solution, pH 6.0. Flow rate was 0 4 ml/min. column temperature was 30° C. UV was recorded at 210 nm. Data acquisition and peak integration was performed using Chromeleon™ 7 (Thermo Scientific).

SEQUENCE LISTING

| SEQ | | |
|---|---|---|
| ID | | |
| NO | Description | Sequence |
| 1 | CH1 of human gamma 1- | ASTKGPSVFPLAPSSKSTSGGTAALGCLVKDYFPEPVTVSWNSGA |
| | type heavy chain | LTSGVHTFPAVLQSSGLYSLSSVVTVPSSSLGTQTYICNVNHKPS |
| | | NTKVDKKV |
| 2 | hinge region of human | EPKSCDKTHTCP |
| | gamma 1-type heavy chain | |
| 3 | CH2 of human gamma 1- | PCPAPELLGGPSVFLFPPKPKDTLMISRTPEVTCVVVDVSHEDPE |
| | type heavy chain | VKFNWYVDGVEVHNAKTKPREEQYNSTYRVVSVLTVLHQDWLNGK |
| | | EYKCKVSNKALPAPIEKTISKAK |
| 4 | CH3 of human gamma 1- | GQPREPQVYTLPPSRDELTKNQVSLTCLVKGFYPSDIAVEWESNG |
| | type heavy chain | QPENNYKTTPPVLDSDGSFFLYSKLTVDKSRWQQGNVFSCSVMHE |
| | | ALHNHYTQKSLSLSPGK |
| 5 | constant region of human | ASTKGPSVFPLAPSSKSTSGGTAALGCLVKDYFPEPVTVSWNSGA |
| | gamma 1-type heavy chain | LTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLM |
| | | ISRTPEVTCVVVDVSHEDPEVKENWYVDGVEVHNAKTKPREEQYN |
| | | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEAL |
| | | HNHYTQKSLSLSPGK |
| 6 | CL of human lambda-type | GQPKANPTVTLFPPSSEELQANKATLVCLISDFYPGAVTVAWKAD |
| | light chain | GSPVKAGVETTKPSKQSNNKYAASSYLSLTPEQWKSHRSYSCQVT |
| | | HEGSTVEKTVAPTECS |
| 7 | CL of human kappa-type | RTVAAPSVFIFPPSDEQLKSGTASVVCLLNNFYPREAKVQWKVDN |
| | light chain | ALQSGNSQESVTEQDSKDSTYSLSSTLTLSKADYEKHKVYACEVT |
| | | HQGLSSPVTKSENRGEC |
| 8 | consensus sequence of | DXEXNPGP |
| | 2A peptides | |
| 9 | consensus sequence of | LXXXGDVEXNPGP |
| | 2A peptides | |
| 10 | 2A peptide of foot-and- | APVKQTLNEDLLKLAGDVESNPGP |
| | mouth disease virus | |
| 11 | 2A peptide of foot-and- | EARHKQKIVAPVKQTLNFDLLKLAGDVESNPGP |
| | mouth disease virus | |
| 12 | 2A peptide of porcine | ATNFSLLKQAGDVEENPGP |
| | teschovirus-1 | |
| 13 | 2A peptide of equine | QCTNYALLKLAGDVESNPGP |
| | rhinitis A virus | |
| 14 | 2A peptide of Thosea | EGRGSLLTCGDVEENPGP |
| | asigna virus | |
| 15 | 2A peptide of Thosea | RAEGRGSLLTCGDVEENPGP |
| | asigna virus | |
| 16 | 2A peptide of | DGFCILYLLLILLMRSGDVETNPGP |
| | Strongylocentrotus | |
| | purpuratus | |
| 17 | 2A peptide of Amphimedon | LLCFMLLLLLSGDVELNPGP |
| | queenslandica | |
| 18 | 2A peptide of Amphimedon | HHFMFLLLLLAGDIELNPGP |
| | queenslandica | |
| 19 | 2A peptide of | WFLVLLSFILSGDIEVNPG |
| | Saccoglossus kowalevskii | |
| 20 | 2A peptide of | KNCAMYMLLLSGDVETNPGP |
| | Branchiostoma floridae | |
| 21 | 2A peptide of | MVISQLMLKLAGDVEENPGP |
| | Branchiostoma floridae | |
| 22 | consensus sequence of | RX(R/K)R |
| | furin recognition site | |
| 23 | furin recognition | RKRR |
| | site RKRR | |
| 24 | furin recognition | RRKR |
| | site RRKR | |
| 25 | furin/2A peptide linker | RKRREARHKQKIVAPVKQTLNFDLLKLAGDVESNPGP |
| | according to the | |
| | invention disclosure | |

1. A nucleic acid product consisting of one or more vector nucleic acids encoding an antibody construct comprising at least three different polypeptide chains,
wherein the antibody construct comprises an antibody heavy chain; and
wherein at least two different polypeptide chains of the antibody construct are encoded within the same open reading frame,
wherein consecutive polypeptide chains encoded within said open reading frame are connected by a peptide linker comprising a 2A peptide.

2. The nucleic acid product according to claim 1 consisting of one vector nucleic acid encoding the antibody construct.

3. The nucleic acid product according to claim 1, wherein the antibody heavy chain is one of the at least two different polypeptide chains of the antibody construct which are encoded within the same open reading frame, and optionally the antibody heavy chain is the N-terminal polypeptide chain encoded within said open reading frame.

4. The nucleic acid product according to claim 1, wherein the antibody construct comprises an antibody light chain binding to the antibody heavy chain, wherein the antibody light chain optionally
is encoded within the open reading frame; and/or
is encoded within the open reading frame together with the antibody heavy chain; and/or
comprises a light chain variable region and the antibody heavy chain comprises a heavy chain variable region, wherein the heavy chain variable region and the light chain variable region form an antigen binding region.

5. The nucleic acid product according to claim 1, wherein the antibody construct comprises a second antibody heavy chain, wherein the second antibody heavy chain optionally
binds to the first antibody heavy chain in the antibody construct, optionally using a knob-into-hole technology, and/or
is encoded within the same open reading frame as the first heavy chain, or is encoded within a second open reading frame different from the first open reading frame encoding the first heavy chain

6. The nucleic acid product according to claim 5, wherein the antibody construct comprises a second antibody light chain binding to the second antibody heavy chain, wherein the second antibody light chain optionally
is encoded within the same open reading frame as the second heavy chain, wherein said open reading frame comprises a peptide linker comprising a 2A peptide which connects the C terminus of the second heavy chain with the N terminus of the second light chain, and/or
comprises a light chain variable region and the second antibody heavy chain comprises a heavy chain variable region, wherein said heavy chain variable region and said light chain variable region form an antigen binding region.

7. The nucleic acid product according to claim 1, wherein each heavy chain of the antibody construct independently has one or more of the following features:
(i) it comprises antibody domains derived from a native human antibody, especially from the γ-type heavy chain of a native human IgG antibody;
(ii) it comprises at least one heavy chain constant domain (CH), in particular at least a CH2 domain or a CH3 domain, especially a CH2 domain and a CH3 domain;
(iii) it is capable of forming a homodimer and/or a heterodimer with another antibody heavy chain;
(iv) it comprises a hinge region;
(v) it comprises a CH1 domain;
(vi) it comprises a heavy chain variable domain (VH); and
(vii) it comprises one or more further polypeptide moieties.

8. The nucleic acid product according to claim 1, wherein each light chain of the antibody construct independently has one or more of the following features:
(i) it comprises antibody domains derived from a native human antibody, especially from the κ- or λ-type light chain of a native human antibody;
(ii) it comprises a light chain constant domain (CL);
(iii) it is capable of forming a heterodimer with an antibody heavy chain;
(iv) it comprises a light chain variable domain (VL); and
(v) it comprises one or more further polypeptide moieties.

9. The nucleic acid product according to claim 1, wherein the first heavy chain of the antibody construct, and in particular each heavy chain of the antibody construct, is encoded with a signal peptide at the N terminus; and/or wherein the first light chain of the antibody construct, and in particular each light chain of the antibody construct, is encoded with a signal peptide at the N terminus.

10. The nucleic acid product according to claim 1, wherein one or more vector nucleic acids encode
(a) within a first open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide; and
(v) a first light chain comprising a first light chain variable region; and
within a second open reading frame, in the direction from N terminus to C terminus,
(i) a signal peptide;
(ii) a second heavy chain comprising a second heavy chain variable region;
(iii) a linker peptide comprising a 2A peptide;
(iv) optionally a signal peptide; and
(v) a second light chain comprising a second light chain variable region;
wherein the first heavy chain variable region and the first light chain variable region form an antigen binding region capable of binding a first antigen; and
wherein the second heavy chain variable region and the second light chain variable region form an antigen binding region capable of binding a second antigen; and
wherein the first heavy chain and the second heavy chain bind to each other using a knob-into-hole technology; or
(b) within the same open reading frame, in the direction from N terminus to C terminus,
(i) a signal peptide;
(ii) a first heavy chain comprising a first heavy chain variable region;
(iii) a linker peptide comprising a 2A peptide;
(iv) optionally a signal peptide;
(v) a first light chain comprising a first light chain variable region;
(vi) a linker peptide comprising a 2A peptide;
(vii) optionally a signal peptide;
(viii) a second heavy chain comprising a second heavy chain variable region;
(ix) a linker peptide comprising a 2A peptide;
(x) optionally a signal peptide; and
(xi) a second light chain comprising a second light chain variable region;
wherein the first heavy chain variable region and the first light chain variable region form an antigen binding region capable of binding a first antigen; and
wherein the second heavy chain variable region and the second light chain variable region form an antigen binding region capable of binding a second antigen; and
wherein the first heavy chain and the second heavy chain bind to each other using a knob-into-hole technology; or
(c) within a first open reading frame, in the direction from N terminus to C terminus,
(i) a signal peptide;
(ii) a first heavy chain comprising a heavy chain variable region;
(iii) a linker peptide comprising a 2A peptide;
(iv) optionally a signal peptide; and
(v) a light chain comprising a light chain variable region; and
wherein the one or more vector nucleic acids encode within a second open
reading frame, in the direction from N terminus to C terminus,
(i) a signal peptide; and
(ii) a second heavy chain; and
wherein the heavy chain variable region of the first heavy chain and the light
chain variable region form an antigen binding region capable of binding a first
antigen; and
wherein the second heavy chain comprises an antigen binding region capable
of binding a second antigen; and
wherein the first heavy chain and the second heavy chain bind to each other
using a knob-into-hole technology; or
(d) within a first open reading frame, in the direction from N terminus to C terminus,
(i) a signal peptide;
(ii) a first heavy chain;
(iii) a linker peptide comprising a 2A peptide;
(iv) optionally a signal peptide; and
(v) a second heavy chain; and
wherein the one or more vector nucleic acids encode within a second open reading frame, in the direction from N terminus to C terminus,
(i) a signal peptide; and
(v) a light chain comprising a light chain variable region; and
wherein the first heavy chain comprises a heavy chain variable region which forms an antigen binding region with the light chain variable region, capable of binding a first antigen; and the second heavy chain comprises an antigen binding region capable of binding a second antigen, or the first heavy chain comprises an antigen binding region capable of binding a first antigen; and the second heavy chain comprises a heavy chain variable region which forms an antigen binding region with the light chain variable region, capable of binding a second antigen; and
wherein the first heavy chain and the second heavy chain bind to each other using a knob-into-hole technology; or
(e) within the open reading frame, in the direction from N terminus to C terminus,
(i) a signal peptide;
(ii) a first heavy chain;
(iii) a linker peptide comprising a 2A peptide;
(iv) optionally a signal peptide;
(v) a second heavy chain comprising a heavy chain variable region;
(vi) a linker peptide comprising a 2A peptide;
(vii) optionally a signal peptide; and
(viii) a light chain comprising a light chain variable region; and
wherein the first heavy chain comprises an antigen binding region capable of
binding a first antigen; and
wherein the heavy chain variable region of the second heavy chain and the
light chain variable region form an antigen binding region capable of binding a
second antigen; and
wherein the first heavy chain and the second heavy chain bind to each other
using a knob-into-hole technology.

11. The nucleic acid product according to claim 1, wherein the peptide linker has one or more of the following features:
(i) it comprises a 2A peptide which is derived from a virus selected from the group consisting of foot-and-mouth disease virus, equine rhinitis A virus, porcine teschovirus-1, and Thosea asigna virus;
(ii) it further comprises a protease recognition site N terminal of the 2A peptide, wherein the protease recognition site in particular is a furin recognition site;
(iii) it comprises an amino acid sequence according to SEQ ID NO: 25.

12. The nucleic acid product according to claim 1, providing for
(i) a more homogeneous cellular production of the polypeptide chains of the antibody construct compared to cellular production of the polypeptide chains of the antibody construct using a nucleic acid product wherein each polypeptide chain of the antibody construct is encoded within a separate open reading frame, wherein optionally the amounts of the mRNAs encoding for the different polypeptide chains of said antibody in the cell do not differ by more than factor 10; and/or
(ii) a higher relative amount of correctly assembled antibody constructs after expression of the polypeptide chains of the antibody construct compared to the relative amount of correctly assembled antibody constructs after expression of the polypeptide chains of the antibody construct using a nucleic acid product wherein each polypeptide chain of the antibody construct is encoded within a separate open reading frame, wherein optionally the relative amount of correctly assembled antibody constructs after expression of the polypeptide chains of the antibody construct is at least 10 percentage points higher than the relative amount of correctly assembled antibody constructs using a nucleic acid product wherein each polypeptide chain is encoded within a separate open reading frame.

13. The nucleic acid product according to claim 1, wherein the open reading frame coding for two or more polypeptide chains of the antibody construct is part of an expression cassette which enables expression of the open reading frame.

14. The nucleic acid product according to claim 1, wherein the vector nucleic acids are plasmids.

15. A host cell comprising the nucleic acid product according to claim 1.

16. The host cell according to claim 15, being a mammalian cell, especially a human or rodent cell, in particular a CHO cell.

17. A method for producing an antibody construct, comprising the steps of
(a) providing a host cell according to claim 15,
(b) cultivating the host cell in a cell culture under conditions which allow for production of the antibody construct,
(c) obtaining the antibody construct from the cell culture, and
(d) optionally processing the antibody construct.

18. The method according to claim 17, wherein
step (c) comprises isolating the antibody construct and/or separating the antibody construct from the remaining components of the cell culture; and/or
step (d) comprises formulating the antibody construct as a pharmaceutical composition.

19. The method according to claim 17, wherein
(i) the polypeptide chains of the antibody product are produced more homogeneously compared to production of the polypeptide chains of the antibody construct using a nucleic acid product wherein each polypeptide chain of the antibody construct is encoded within a separate open reading frame, wherein optionally the amounts of the mRNAs coding for the different polypeptide chains of said antibody construct in the cell do not differ by more than factor 10; and/or
(ii) the relative amount of correctly assembled antibody constructs is higher compared to production of the antibody construct using a nucleic acid product wherein each polypeptide chain of the antibody construct is encoded within a separate open reading frame, wherein optionally the relative amount of correctly assembled antibody constructs is at least 10 percentage points higher than the relative amount of correctly assembled antibody constructs using a nucleic acid product wherein each polypeptide chain is encoded within a separate open reading frame.

20. Use of the nucleic acid product according to claim 1 for the production of an antibody construct.

21. A method for producing a host cell comprising the nucleic acid product according to claim 1, comprising introducing the nucleic acid product according to claim 1 into a host cell.

22. Use of the host cell according to claim 15 for the production of an antibody construct.
